(19) United States
(12) Patent Application Publication
(10) Pub. No.: US 2015/0101072 A1
(43) Pub. Date: Apr. 9, 2015

(54) COMBINING FUSARIUM 2 RESISTANCE GENE (FON2) AND RED FLESH IN WATERMELON

(71) Applicant: Brenda LANINI, Davis, CA (US)

(72) Inventor: Brenda LANINI, Davis, CA (US)

(73) Assignee: Vilmorin & Cie, Paris (FR)

(21) Appl. No.: 14/507,277

(22) Filed: Oct. 6, 2014

Related U.S. Application Data
(60) Provisional application No. 61/886,877, filed on Oct. 4, 2013.

(51) Int. Cl. A01H 5/08; A01H 1/02; A01H 4/00

(52) U.S. Cl. CPC A01H 5/08; A01H 4/008; A01H 1/02
USPC 800/265; 800/308; 435/430; 435/410

(57) ABSTRACT

The present invention provides watermelon plants with both resistance to Fusarium oxysporum and desirable agronomic traits, such as desirable fruit traits. The present invention also provides methods of making such plants and methods of using such plants to produce additional watermelon plants.

### CROSS-REFERENCE TO RELATED APPLICATIONS

This application claims priority to, and the benefit of U.S. Provisional Patent Application Ser. No. 61/886,877, which is herein incorporated by reference in its entirety.

### FIELD OF THE INVENTION

The present invention relates to plant breeding. More specifically, the present invention relates to Fusarium wilt resistant watermelon plants with sweet, red fleshed fruit. The invention further describes methods for breaking of the genetic linkage between Fusarium oxysporum f sp. niveum Race 2 (Fon2) resistance, and undesirable white flesh fruit traits associated with PI watermelon lines.

### BACKGROUND OF THE INVENTION

Cucurbita Citrullus lanatus, (commonly known as watermelon) is a plant native to southern Africa, believed to have originated in areas near Namibia, Botswana, and Zimbabwe (Wein, H. C. 1997. “The Cucurbits: Cucumber, Melon, Squash and Pumpkin” The Physiology of Vegetable Crops. CAB International Ch. 9). Thanks to its sweet red-fleshed fruit, watermelon has become a popular summer food throughout the world. According to Food and Agriculture Organization (FAO, 2011), world production of watermelon exceeded 104 million tons.

A variety of pathogens affect the productivity of watermelon plants including virus, fungi, bacteria, nematodes, and insects (Larson et al., 2000 “Florida Crop/Pest Management Profile: Watermelon” Agronomy, Florida Cooperative Extension Service CIR1236). Fusarium wilt in particular has been an important factor in U.S. watermelon production since the late 1800's, with economic losses in heavily infested fields of resistant watermelons capable of reaching 100% crop loss.

At present, there are three described isolates (races) of Fusarium oxysporum f. sp. niveum (Fon) affecting watermelon in the United States: races 0, 1, and 2 (Cirulli et al. 1972, “Variation in pathogenicity in Fusarium oxysporum f. sp. niveum and resistance in watermelon cultivars” Actas Congr Union Fitopathol Mediter, Oeiras, 3^{rd} p 491-500; and Martyn et al., 1987, “Fusarium oxysporum f. sp. niveum, race 2: A highly aggressive race new to the United States” Plant Disease 71:233-236.), each of which is herein incorporated by reference in its entirety.

Once infested, fields retain active levels of F. oxysporum for many years and severely limit watermelon production. Attempts to control Fusarium have included long term crop rotation, soil solarization, and fumigation, among others (Martyn and Hartz, 1986 “Use of soil solarization to control Fusarium wilt of watermelon” Plant Dis. 70:762-766; Hopkins and Elmstrom, 1979 “Evaluation of soil fumigants and application methods for the control of Fusarium wilt of watermelon. Plant Dis. Rptr. 63:1003-1006), each of which is herein incorporated by reference in its entirety. Despite all these options, the most effective and efficient means for control of Fusarium wilt has been through the use of genetically resistant varieties of watermelons.

Over the years, many watermelon cultivars resistant to Fusarium wilt Fon0 and Fon1 have been released from breeding programs starting with W. A. Orton in 1907, and leading to more contemporary diploid and triploid (seedless) “commercial” lines such as “Fiesta” (Syngenta), “Summer Flavor 790” (Abbott & Cobb), and “Afternoon Delight” (Dwayne Palmer) (Orton, Wash. 1907 “On methods of breeding for disease-resistance” Proc. Soc. Hort. Sci. 5:28; for a more complete table of commercial watermelon lines and their resistance to Fon1, please see “Midwest Vegetable Production Guide for Commercial Growers 2013” ID-56 pg 97). However, as the prevalence of Fon2 Fusarium wilt has increased throughout the US east coast, many of these cultivars have succumbed to wilt.

The increasing susceptibility of currently available “resistant” watermelon lines has created a need for new sources of resistance. One such source was published in a 1991 article describing a PI-236341 Citrullus line collected from the Republic of South Africa by the Department of Agrucultural Technical services, and found to exhibit resistance to all three Fusarium oxysporum f. sp. niveum races, Fon0, 1, and 2 (Martyn, R. D. 1991. “Resistance to Races 0, 1, and 2 of Fusarium Wilt of Watermelon in Citrullus sp. PI-296341-FR” Hort. Science 26(4):429-432, incorporated herein by reference in its entirety). Unfortunately, while the PI-236341 line could be bred to reliably inherit Fon2 resistance, its fruit was a small (500 to 1200 g), grayish-green watermelon, with white, non-sweet flesh. Moreover, these negative fruit character traits (hard, white flesh, non-sweet) were discovered to be genetically linked to Fon2 resistance, making PI-296341 unusable for commercial breeding programs.

In an attempt to integrate Fon2 resistance to commercial lines, some growers have begun using grafting techniques to combine PI-296341 and other Fon2 resistant root stocks with more commercially viable scions (Huh Y C, and Om Y H, 2002 “Utilization of Citrullus Germplasm with Resistance to Fusarium Wilt (Fusarium oxysporum f. sp. niveum) for Watermelon Rootstocks” ISHS Acta Horticulturae 588). This approach however, is expensive and time-consuming and must be repeated each growing season as watermelon plants are an annual species. Thus there is a real need for a “breeder level” Fon2 resistant watermelon.

### BRIEF SUMMARY OF THE INVENTION

The present invention provides watermelon plants, plant parts, and plant seeds. In some embodiments, the watermelon plants are resistance to at least one F. oxysporum f. sp. niveum (Fon) race. In some embodiments, the watermelon plants are at least resistant to Fon race 2. In some embodiments, the watermelon plants have commercially acceptable fruit quality.

In some embodiments, the watermelon plants are also resistant to F. oxysporum f. sp. niveum (Fon) race 0, Fon race 1, and or Fon race 3.

In some embodiments, the watermelon plants have red or pink fruit flesh.

In some embodiments, the watermelon plants have fruit flesh that is sweet. In some embodiments, the fruit flesh has a Brix solid soluble content greater than 3, 4, 5, 6, 7, or more.

In some embodiments, the watermelon plants triploidy or tetraploidy plants.

In some embodiments, the watermelon plants contain a Fon2 resistance allele derived from PI296341, PI482246, PI482252, PI296335, PI1271769, PI 255136, PI 270564, PI271769, USVL246-FR2, USVL252-FR2, and USVL335-FR2.

In some embodiments, the watermelon plants have an ultra-firm watermelon flesh phenotype.

The present invention provides methods of culturing plant tissue, plant part, plant organ, or cell culture comprising culturing at least part of the watermelon plants of the present invention. In some embodiments, said plant tissue, plant part, plant organ, or cell culture is cultured in conditions conducive to plant regeneration.

The present invention provides plant parts of the watermelon plants of the present invention. In some embodiments, the part is selected from the group consisting of pollen, an ovule, a leaf, an embryo, a root, a root tip, an anther, a flower, a fruit, a stem, a shoot, a seed, a protoplast, a cell, a callus, and a scion.

The present invention provides methods of producing watermelon plants. In some embodiments, the methods comprise crossing the watermelon plant of the present invention with another plant. In some embodiments, the plants produced by the methods are triploid watermelon plants.

The present invention provides methods of breeding watermelon plants. In some embodiments, the methods are used to produce altered pathogen tolerance and/or resistance while having commercially acceptable fruit quality. In some embodiments, the methods comprise (i) making a cross between the watermelon plant of the present invention with a second watermelon plant to produce a F1 plant. In some embodiments, the methods further comprise (ii) backcrossing the F1 plant to the second plant. In some embodiments, the methods further comprise (iii) repeating the backcrossing step one or more times to generate progeny plants. In some embodiments, the progeny plants have conferred or enhanced Fon2 tolerance and/or resistance compared to that of the second plant prior to breeding, and have commercially acceptable fruit quality.

The present invention provides watermelon seeds of a watermelon line, wherein representative seed of said line is having been deposited under NCIMB Accession No: ______. The present invention provides a plant of watermelon line, wherein representative seed of said line is having been deposited under NCIMB Accession No: ______. The present invention provides a pollen, an ovule, a fruit of said plant. In some embodiments, the fruit is produced by self-pollination of the plant.

The present invention provides watermelon plants, or a part thereof, having all the physiological and morphological characteristics of the watermelon plant grown from the seed deposited under NCIMB Accession No: ______.

The present invention provides tissue culture of cells produced from the plant grown from the seed deposited under NCIMB Accession No: ______.

The present invention provides watermelon plants regenerated from the tissue culture of the present invention, wherein the regenerated plant has all the morphological and physiological characteristics of the watermelon plant grown from the seed deposited under NCIMB Accession No: ______.

The present invention provides methods for producing a watermelon fruit. In some embodiments, the method comprises allowing pollination of a first watermelon plant and a second watermelon plant, wherein the first watermelon plant is the watermelon plant grown from the seed deposited under NCIMB Accession No: ______. A watermelon seed or fruit produced by the method, and watermelon plants, or part thereof, produced by growing said seed are also parts of the present invention.

The present invention provides methods for producing seeds of a watermelon plant, wherein the methods comprise the steps of: a) growing in a field the watermelon plant from the seed deposited under NCIMB Accession No: ______ or a plant having physiological and morphological characteristics of said watermelon plant. In some embodiments, the methods further comprise b) conducting pollination of said plant. In some embodiments, the methods further comprise c) harvesting seed of said plant.

The present invention provides methods for producing a hybrid watermelon variety. In some embodiments, the methods comprise (a) planting in a field a first and a second watermelon plant, wherein said first watermelon plant is the male parent, wherein said second watermelon plant is the female parent, and wherein said first or said second watermelon plant is the watermelon plant grown from the seed deposited under NCIMB Accession No: ______ or a plant having all physiological and morphological characteristics of said watermelon plant. In some embodiments, the methods further comprise (b) conducting pollination between said first and second watermelon plants. In some embodiments, the methods further comprise (c) harvesting seed from said female parent, wherein said seed is seed of a hybrid watermelon variety. In some embodiments, the methods further comprise identifying plants resistant to F. oxysporum f. sp. niveum (Fon) race 2. In some embodiments, the methods further comprise identifying plants having commercially acceptable fruit quality.

The present invention provides methods for producing a watermelon plants that contain in their genetic material one or more transgenes. In some embodiments, the methods comprise crossing the watermelon plant grown from the seed deposited under NCIMB Accession No: ______ or a plant having all physiological and morphological characteristics of said watermelon plant with either a second plant of another watermelon line which contains a transgene, or a transformed watermelon plant derived from the plant grown from the seed deposited under NCIMB Accession No: ______ or a plant having all physiological and morphological characteristics of said watermelon plant, so that the genetic material of the progeny that results from the cross contains the transgene(s) operably linked to a regulatory element. In some embodiments, the transgene is selected from the group consisting of male sterility, male fertility, herbicide resistance, insect resistance, disease resistance, water stress tolerance, and increased size, weight, sweetness, and flesh firmness.

The present invention provides methods for introducing one or more desired traits into the watermelon plant grown from the seed deposited under NCIMB Accession No: ______ or a plant having all physiological and morphological characteristics of said watermelon plant. In some embodiments, the methods comprise (a) crossing the watermelon plant grown from the seed deposited under NCIMB Accession No: ______ or a plant having all physiological and morphological characteristics of said watermelon plant with plants of another watermelon line that comprise one or more desired traits to produce progeny plants, In some embodiments, the methods further comprise (b) selecting progeny plants that have the one or more desired traits to produce selected progeny plants. In some embodiments, the methods further comprise (c) crossing the selected progeny plants with the watermelon plant grown from the seed deposited under NCIMB Accession No: ______ or a plant having all physiological and morphological characteristics of said watermelon plant, or crossing the selected progeny plants with the other watermelon line that comprise one or more desired traits to produce progeny plants to produce backcross progeny plants. In some embodiments, the methods further comprise (d) selecting for backcross progeny plants that have said one or more desired traits and physiological and morphological characteristics of the parental watermelon plant to produce selected backcross progeny plants. In some embodiments, the methods further comprise (e) repeating steps (c) and (d) one or more times in succession to produce selected second or higher backcross progeny plants that comprise the desired one or more trait and the physiological and morphological characteristics of the watermelon plant.

The present invention also provides watermelon plant progenies. In some embodiments, the watermelon plant progenies are produced from the seeds of the present invention. In some embodiments, the watermelon plant progeny are resistance to F. oxysporum f. sp. niveum (Fon) race 2, and have commercially acceptable fruit quality. In some embodiments, the watermelon progeny plants have one or more or all physiological and morphological characteristics of the watermelon plants grown from the seed deposited under NCIMB Accession No: ______.

### BRIEF DESCRIPTION OF THE FIGURES

FIG. 1 depicts an example of early susceptible reaction with ‘Black Diamond’.

FIG. 2 depicts an example of resistant reaction from ‘Dixie Lee” to race 1.

### DETAILED DESCRIPTION

### Definitions

While the following terms are believed to be well understood by one of ordinary skill in the art, the following definitions are set forth to facilitate explanation of the presently disclosed subject matter

The term “a” or “an” refers to one or more of that entity; for example, “a gene” refers to one or more genes or at least one gene. As such, the terms “a” (or “an”), “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used herein, the verb “comprise” as is used in this description and in the claims and its conjugations are used in its non-limiting sense to mean that items following the word are included, but items not specifically mentioned are not excluded.

As used herein the term “watermelon” or “watermelon plant” refers to a plant of the genus Citrullus, specifically to plants of Citrullus lanatus.

As used herein, the term “Fusarium wilt” refers to a disease caused by Fusarium oxysporum, specifically to any race of Fusarium oxysporum f. sp. niveum known to infect watermelon plants. Plants infected with the fungus will generally show symptoms of stunted growth, loss of turgor pressure, and wilting of leaves and stems. The disease progression is also associated with the yellowing and eventual necrosis of the plant.

As used herein, the term “pathogen” refers to an agent that causes disease, especially a living microorganism such as an insect, a bacterium, virus, nematode or fungus.

As used herein, the term “resistant”, or “resistance”, with regards to Fusarium wilt describes a plant, line or cultivar that shows no, fewer or reduced symptoms to a biotic pest or pathogen than a susceptible (or more susceptible) plant, line or variety to that biotic pest or pathogen. These terms are variously applied to describe plants that show no symptoms as well as plants showing some symptoms but that are still able to produce marketable product with an acceptable yield. Some lines that are referred to as resistant are only so in the sense that they may still produce a crop, even though the plants may appear visually stunted and the yield is reduced compared to uninfected plants. As defined by the International Seed Federation (ISF), a non-governmental, non-profit organization representing the seed industry (see “Definition of the Terms Describing the Reaction of Plants to Pests or Pathogens and to Abiotic Stresses for the Vegetable Seed Industry”, May 2005), the recognition of whether a plant is affected by or subject to a pest or pathogen can depend on the analytical method employed. Resistance is defined by the ISF as the ability of plant types to restrict the growth and development of a specified pest or pathogen and/or the damage they cause when compared to susceptible plant varieties under similar environmental conditions and pest or pathogen pressure. Resistant plant types may still exhibit some disease symptoms or damage. Two levels of resistance are defined. The term “high/standard resistance” is used for plant varieties that highly restrict the growth and development of the specified pest or pathogen under normal pest or pathogen pressure when compared to susceptible varieties. “Moderate/intermediate resistance” is applied to plant types that restrict the growth and development of the specified pest or pathogen, but exhibit a greater range of symptoms or damage compared to plant types with high resistance. Plant types with intermediate resistance will show less severe symptoms than susceptible plant varieties, when grown under similar field conditions and pathogen pressure. Methods of evaluating resistance are well known to one skilled in the art. Such evaluation may be performed by visual observation of a plant or a plant part (e.g., leaves, roots, flowers, fruits et. al) in determining the severity of symptoms. For example, when each plant is given a resistance score on a scale of 1 to 5 based on the severity of the reaction or symptoms, with 1 being the resistance score applied to the most resistant plants (e.g., no symptoms, or with the least symptoms), and 5 the score applied to the plants with the most severe symptoms, then a line is rated as being resistant when at least 75% of the plants have a resistance score at a 1, 2, or 3 level, while susceptible lines are those having more than 25% of the plants scoring at a 4 or 5 level. If a more detailed visual evaluation is possible, then one can use a scale from 1 to 10 so as to broaden out the range of scores and thereby hopefully provide a greater scoring spread among the plants being evaluated. One such method for evaluation can be conducted for example by dipping the roots of young seedlings into a Fusarium containing culture and then measuring the quantity of wilting as is described in (Martyn, R. D. 1987 “Fusarium oxysporum f sp. niveum race 2: A highly aggressive race new to the United States” Plant Dis. 71:233-236). In some embodiments, the degree of resistance scoring is performed at 1-7 days, 7-14 days, 14-18 days, or more after inoculation. In some embodiments, a plant is determined to be susceptible when (1) the cotyledons begin to wilt and turn yellowish approximately 5 days after inoculation; (2) root system is usually dead and plant can be easily pulled from soil by a slight tug; and/or (3) plants often die out. In some embodiments, a plant is determined to be resistant when the plant grows normally and remains lush green throughout the test. A plant resistant to the virus has a root system that continues to grow after inoculation. In some embodiments, a plant is determined to have intermediate resistance, when the test is not very severe and some plants are only slightly affected by the disease. In a plant having intermediate resistance, a new root is sometimes generated from the base of a plant after transplanting while the primary root has died. As used herein, the term “susceptible” with regards to Fusarium wilt refers to a plant having no or virtually no resistance to the pathogen resulting in entry of the pathogen into the plant and multiplication and systemic spread of the pathogen, resulting in disease symptoms. The term “susceptible” is therefore equivalent to “non-resistant”.

As used herein, the term “genotype” refers to the genetic makeup of an individual cell, cell culture, tissue, organism (e.g., a plant), or group of organisms.

As used herein, the term “allele(s)” means any of one or more alternative forms of a gene, all of which alleles relate to at least one trait or characteristic. In a diploid cell, the two alleles of a given gene occupy corresponding loci on a pair of homologous chromosomes. Since the present invention relates to QTLs, i.e. genomic regions that may comprise one or more genes or regulatory sequences, it is in some instances more accurate to refer to “haplotype” (i.e. an allele of a chromosomal segment) instead of “allele”, however, in those instances, the term “allele” should be understood to comprise the term “haplotype”. Alleles are considered identical when they express a similar phenotype. Differences in sequence are possible but not important as long as they do not influence phenotype.

As used herein, the term “locus” (loci plural) means a specific place or places or a site on a chromosome where for example a gene or genetic marker is found.

As used herein, the term genetically linked refers to two or more traits that are co-inherited at a high rate during breeding such that they are difficult to separate through crossing.

A “recombination” or “recombination event” as used herein refers to a chromosomal crossing over or independent assortment. The term “recombinant” refers to a plant having a new genetic make up arising as a result of recombination event.

As used herein, the term “molecular marker” or “genetic marker” refers to an indicator that is used in methods for visualizing differences in characteristics of nucleic acid sequences. Examples of such indicators are restriction fragment length polymorphism (RFLP) markers, amplified fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Mapping of molecular markers in the vicinity of an allele is a procedure which can be performed quite easily by the average person skilled in molecular-biological techniques which techniques are for instance described in Lefebvre and Chevre, 1995; Lorez and Wenzel, 2007, Srivastava and Narula, 2004, Meksem and Kahl, 2005, Phillips and Vasil, 2001. General information concerning AFLP technology can be found in Vos et al. (1995, AFLP: a new technique for DNA fingerprinting, Nucleic Acids Res. 1995 November 11; 23(21): 4407-4414).

As used herein, the term “trait” refers to characteristic or phenotype. For example, in the context of the present invention “resistance” and “susceptibility” relates to the symptoms observed on a plant infected with the fungus L. taurica or with a potyvirus as described herein. A trait may be inherited in a dominant or recessive manner, or in a partial or incomplete-dominant manner A trait may be monogenic (i.e. determined by a single locus) or polygenic (i.e. determined by more than one locus) or may also result from the interaction of one or more genes with the environment. A dominant trait results in a complete phenotypic manifestation at heterozygous or homozygous state; a recessive trait manifests itself only when present at homozygous state.

As used herein, the term “homozygous” means a genetic condition existing when two identical alleles reside at a specific locus, but are positioned individually on corresponding pairs of homologous chromosomes in the cell of a diploid organism. Conversely, as used herein, the term “heterozygous” means a genetic condition existing when two different alleles reside at a specific locus, but are positioned individually on corresponding pairs of homologous chromosomes in the cell of a diploid organism.

As used herein, the term “plant” includes the whole plant or any parts or derivatives thereof, such as plant cells, plant protoplasts, plant cell tissue cultures from which watermelon plants can be regenerated, plant calli, embryos, pollen, ovules, fruit, flowers, leaves, seeds, roots, root tips and the like.

As used herein, the term “phenotype” refers to the observable characters of an individual cell, cell culture, organism (e.g., a plant), or group of organisms which results from the interaction between that individual's genetic makeup (i.e., genotype) and the environment.

As used herein, the term “derived from” refers to the origin or source, and may include naturally occurring, recombinant, unpurified, or purified molecules. A nucleic acid or an amino acid derived from an origin or source may have all kinds of nucleotide changes or protein modification as defined elsewhere herein.

As used herein, the term “offspring” refers to any plant resulting as progeny from a vegetative or sexual reproduction from one or more parent plants or descendants thereof. For instance an offspring plant may be obtained by cloning or selling of a parent plant or by crossing two parents plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

As used herein, the term “cross”, “crossing”, “cross pollination” or “cross-breeding” refer to the process by which the pollen of one flower on one plant is applied (artificially or naturally) to the ovule (stigma) of a flower on another plant.

As used herein, the term “cultivar” refers to a variety, strain or race of plant that has been produced by horticultural or agronomic techniques and is not normally found in wild populations.

As used herein, the terms “dicotyledon,” “dicot” and “dicotyledonous” refer to a flowering plant having an embryo containing two seed halves or cotyledons. Examples include tobacco; tomato; the legumes, including peas, alfalfa, clover and soybeans; oaks; maples; roses; mints; squashes; daisies; walnuts; cacti; violets and buttercups.

### Plant Diseases Resistance

Plant disease resistance derives both from pre-formed defenses and from infection-induced responses mediated by the plant immune system. Disease outcome is determined by the three-way interaction of the pathogen, the plant, and the environmental conditions (an interaction known as the disease triangle). Defense-activating compounds can move cell-to-cell and systemically through the plant vascular system, but plants do not have circulating immune cells so most cell types in plants retain the capacity to express a broad suite of antimicrobial defenses. Although obvious qualitative differences in disease resistance can be observed when some plants are compared (allowing classification as “resistant” or “susceptible” after infection by the same pathogen strain at similar pathogen pressure in similar environments), a gradation of quantitative differences in disease resistance is more typically observed between plant lines or genotypes.

Preformed structures and compounds that contribute to resistance in plants include, but are not limited to, plant cuticle/surface, plant cell walls, antimicrobial chemicals (e.g., glucosides, saponins), antimicrobial proteins, enzyme inhibitors, detoxifying enzymes that break down pathogen-derived toxins, receptors that perceive pathogen presence and active inducible plant defenses. Inducible plant defenses that are generated upon or after infection include, but are not limited to, cell wall reinforcement (e.g., increased callose, lignin, suberin, cell wall proteins), antimicrobial chemicals (e.g., reactive oxygen species such as hydrogen peroxide, peroxynitrite, or complex phytoalexins such as genistein or camalexin), antimicrobial proteins (e.g., defensins, thionins, or pathogenesis-related (PR) proteins), antimicrobial enzymes (e.g., chitinases, beta-glucanases, peroxidases), hypersensitive response (e.g., rapid host cell death response associated with defense mediated by resistance genes), and post-translation gene silencing.

Plant immune systems show some mechanistic similarities and apparent common origin with the immune systems of insects and mammals, but also exhibit many plant-specific characteristics. As in most cellular responses to the environment, defenses are activated when receptor proteins directly or indirectly detect pathogen presence and trigger ion channel gating, oxidative burst, cellular redox changes, protein kinase cascades, and/or other responses that either directly activate cellular changes (such as cell wall reinforcement), or activate changes in gene expression that then elevate plant defense responses.

Plants, like animals, have a basal immune system that includes a small number of pattern recognition receptors that are specific for broadly conserved microbe-associated molecular patterns (MAMPs, also called pathogen-associated molecular patterns or PAMPs). Examples of these microbial compounds that elicit plant basal defense include bacterial flagellin or lipopolysaccharides, or fungal chitin. The defenses induced by MAMP perception are sufficient to repel most potentially pathogenic microorganisms. However, pathogens express effector proteins that are adapted to allow them to infect certain plant species; these effectors often enhance pathogen virulence by suppressing basal host defenses.

Importantly, plants have evolved R genes (resistance genes) whose products allow recognition of specific pathogen effectors, either through direct binding of the effector or by recognition of the alteration that the effector has caused to a host protein. R gene products control a broad set of disease resistance responses whose induction is often sufficiently rapid and strong to stop adapted pathogens from further growth or spread. Plant genomes each contain a few hundred apparent R genes, and the R genes studied to date usually confer specificity for particular strains of a pathogen species. As first noted by Harold Flor in the mid-20th century in his formulation of the gene-for-gene relationship, the plant R gene and the pathogen “avirulence gene” (effector gene) must have matched specificity for that R gene to confer resistance. The presence of an R gene can place significant selective pressure on the pathogen to alter or delete the corresponding avirulence/effector gene. Some R genes show evidence of high stability over millions of years while other R genes, especially those that occur in small clusters of similar genes, can evolve new pathogen specificities over much shorter time periods.

The use of receptors carrying leucine-rich repeat (LRR) pathogen recognition specificity domains is common to plant, insect, jawless vertebrate and mammal immune systems, as is the presence of Toll/Interleukin receptor (TIR) domains in many of these receptors, and the expression of defensins, thionins, oxidative burst and other defense responses (Jones and Dangl. 2006 The plant immune system. Nature 444:323-329. Ting et al. 2008. NLRs at the intersection of cell death and immunity. Nat Rev Immunol. 8:372-379. which are incorporated herein by reference in their entireties).

Some of the key endogenous chemical mediators of plant defense signal transduction include salicylic acid, jasmonic acid or jasmonate, ethylene, reactive oxygen species, and nitric oxide. Numerous genes and/or proteins have been identified that mediate plant defense signal transduction (Hammond-Kosack and Parker, 2003, Deciphering plant-pathogen communication: fresh perspectives for molecular resistance breeding. Curr. Opin. Biotechnol. 14:177-193). Cytoskeleton and vesicle trafficking dynamics help to target plant defense responses asymmetrically within plant cells, toward the point of pathogen attack.

Plant immune systems can also respond to an initial infection in one part of the plant by physiologically elevating the capacity for a successful defense response in other parts of the plant. These responses include systemic acquired resistance, largely mediated by salicylic acid-dependent pathways, and induced systemic resistance, largely mediated by jasmonic acid-dependent pathways. Against viruses, plants often induce pathogen-specific gene silencing mechanisms mediated by RNA interference. These are primitive forms of adaptive immunity.

In a small number of cases, plant genes have been identified that are broadly effective against an entire pathogen species (against a microbial species that is pathogenic on other genotypes of that host species). Examples include barley MLO against powdery mildew, wheat Lr34 against leaf rust, and wheat Yr36 against stripe rust. An array of mechanisms for this type of resistance may exist depending on the particular gene and plant-pathogen combination. Other reasons for effective plant immunity can include a relatively complete lack of co-adaptation (the pathogen and/or plant lack multiple mechanisms needed for colonization and growth within that host species), or a particularly effective suite of pre-formed defenses.

Resistance to disease varies among plants. It may be either total (a plant is immune to a specific pathogen) or partial (a plant is tolerant to a pathogen, suffering minimal injury). The two broad categories of resistance to plant diseases are vertical (specific) and horizontal (nonspecific). A plant variety that exhibits a high degree of resistance to a single race, or strain, of a pathogen is said to be vertically resistant; this ability usually is controlled by one or a few plant genes. Horizontal resistance, on the other hand, protects plant varieties against several strains of a pathogen, although the protection is not as complete. Horizontal resistance is more common and involves at least several or many genes.

Several means of obtaining disease-resistant plants are commonly employed alone or in combination. These include, but are not limited to, introduction from an outside source, selection, and induced variation. All three may be used at different stages in a continuous process; for example, varieties free from injurious insects or plant diseases may be introduced for comparison with local varieties. The more promising lines or strains are then selected for further propagation, and they are further improved by promoting as much variation as possible through hybridization or special treatment. Finally, selection of the plants showing greatest promise takes place.

Methods used in breeding plants for disease resistance are similar to those used in breeding for other characters. It is necessary to know as much as possible about the nature of inheritance of the resistant characters in the host plant and the existence of physiological races or strains of the pathogen.

Various species of fungi, viruses, and bacteria cause destructive diseases of watermelon, including but are not limited to anthracnose (Colletotrichum lagenarium (Pass.) Ellis & Halst), downy mildew (Pseudoperonospora cubensis Berk. & M. A. Curtis), Fusarium wilt (Fusarium oxysporum Schlechtend.: Fr. f. sp. niveum (E.F. Sm.) W. C. Snyder & H. N. Hans), gummy stem blight (Didymella bryoniae (Auersw.) Rehm), Monosporascus root rot and vine decline (Monosporascus cannonballus Pollack & Uecker), Phytophthora blight (Phytophthora capsici Leonian), Pythium damping-off (Pythium spp.), powdery mildew (Podosphaera xanthii (Castagne) U. Braun & N. Shishkoff), cucumber mosaic (CMV), papaya ringspot (PRSV type W, previously known as WMV), watermelon mosaic (WMV; previously known as WMV-2), squash mosaic (SqMV), zucchini yellow mosaic (ZYMV), watermelon vine decline virus, bacterial fruit blotch caused by Acidovorax avenae subsp. citrulli Schaad et al. The genetics of resistance have been described for the control of Fusarium wilt, gummy stem blight, anthracnose, watermelon mosaic, and zucchini yellow mosaic (Guner, N., and T. C. Wehner. 2003. Gene list for watermelon. Cucurbit Genetics Cooperative Report 26:76-92; Xu et al., 2004. Inheritance of resistance to zucchini yellow mosaic virus in watermelon. Journal of Heredity 95:498-502).

Watermelon plants resistance to various pathogens, including but are not limited to Angular Leaf Spot, Alternaria, Anthracnose, Bacterial Wilt, Black Rot, Black Spot, Cucumber Mosaic Virus, Cucumber Vein Yellowing virus, Downy Mildew, fusarium Fruit Rot, Fusarium Wilt, Gummy stem blight, Powdery Mildew, Phytophthora Fruit Rot, Papaya Ringspot Virus, Root Rot, Scab, Stemphyllium, Target Leaf Spot, Watermelon mosaic Virus, Zucchini Yellow Mosaic Virus, are made by several breeding companies such as Fedco, Harris Seeds, High Mowing Organic, Holmes, Johnny's, Rupps, Seedway, Sieger, Stokes, Takii, and Territorial, see Watermelon: Disease Resistance Table (Cornell University Vegetable MD Online, April 2012), Norton et al. (1995. ‘AU-Sweet Scarlet’ watermelon. HortScience 30:393-394), Provvidenti, R. (1991. Inheritance of resistance to the Florida strain of zucchini yellow mosaic virus in watermelon. HortScience 26:407-408), Xu et al. (2004. Inheritance of resistance to zucchini yellow mosaic virus in watermelon. Journal of Heredity 95:498-502),

Several genes for resistance to pathogens have been isolated, including, but not limited to genes for resistance to the red pumpkin beetle (Aulacophora foveicollis LLucas), Af: resistance to the fruit fly (Dacus cucurbitae Coqhillett), (Khandelwal et al., Canadian Journal of Genetics and Cytology, 20:31-34; Vashistha et al., Proceedings of 3^{rd} International symposium on Sub-Tropical and Tropical Horticulture: 75-81), and pm gene for susceptibility to powdery mildew (Robinson, et al., 1975. Inheritance of susceptibility to powdery mildew in the watermelon. Journal of Heredity 66:310-311.), each of which is incorporated by reference herein in entirety for all purposes.

Watermelon varieties resistant to Fusarium wilt have been identified. ‘Black Diamond’ and ‘Sugar Baby’ are susceptible to all races, ‘Quetzali’ and ‘Mickylee’ are resistant to race 0, ‘Charleston Gray’ is resistant to race 0 and moderately resistant to race 1, ‘Calhoun Gray’ is resistant to races 0 and 1, and PI 296341 and PI 271769 are resistant to all races (Maynard, D. N., ed. 2001. Watermelons. Characteristics, production, and marketing. I ed., Alexandria, Va.: ASHS Press. 227 pp.). In addition, the inheritance of resistance to race 1 has been described. Resistance was inherited as a single dominant gene (Fo-1) in crosses of the resistant ‘Calhoun Gray’ or ‘Summit’ with the susceptible ‘NH Midget’ (Henderson et al., 1970. The inheritance of Fusarium wilt resistance in watermelon, Citrullus lanatus (Thunb.) Mansf. Proceedings of American Society for Horticultural Science 95:276-282.). Each of the references cited is incorporated by reference herein in entirety for all purposes.

### Fusarium Oxysporum

Fusarium wilt is a plant disease caused by a Fusarium oxysporum fungal parasite commonly found in the micro flora of soil. The fungus is one of the most common fungi isolated from asymptomatic roots of crop plants. That saprophytic isolates of Fusarium oxysporum do not cause disease is likely due to their incompatibility or inability to enter the vascular tissue (Gao et al 1995. “The rate of vascular colonization as a measure of the genotypic interaction between various cultivars of tomato and various formae specials” Physiol Mol Plant Pathol 46:29-43). Strains that enter into the parasitic phase make their was through the root and xylem elements to become full fledged parasites (Martyn R. D. 2012. “Fusarium wilt of watermelon: a historical review” Proc. Of the 10^{th} EUCARPIA meeting on genetics October 15-18^{th}). Fusarium infection can occur at any stage of plant growth. For seedlings, damping-off may cause rot in the hypocotyls leading to stunted growth. Infection in mature plants can cause a loss of turgor pressure and wilting, usually followed by a yellowing and necrosis of the leaves. A “one-sided wilt” is a common symptom in which one or more shoots show disease symptoms while the others remain unaffected.

The disease is not spread above-ground but instead through fungal spores in the soil. The fungus can be introduced from any contaminated material including compost, farming tools, and contaminated seeds. Once infested, fields retain active levels of F. oxysporum for many years and severely limit field production. Attempts to control Fusarium have included long term crop rotation, soil solarization, and fumigation, among others (Martyn and Hartz, 1986 “Use of soil solarization to control Fusarium wilt of watermelon” (Plant Dis. 70:762-766; Hopkins and Elmstrom, 1979 “Evaluation of soil fumigants and application methods for the control of Fusarium wilt of watermelon).

While collectively, F. oxysporum has a broad host range of plants, individual isolates of the fungus however have been found to cause disease on only a narrow range of plant species. This observation of host-pathogen specificity has led to the further subclassification of F. oxysporum isolates into “special forms” or formae speciales (f. sp.) that denote their preferred host. For example, isolates with host specificity to the common cucumber are referred to as Fusarium oxysporum Esp. cucumerinum (Foc) while isolates infecting musk melons are denoted as Fusarium oxysporum f. sp. melonis (Fom). As additional isolates for a particular species are discovered, they are assigned a new sequential race number at the end of their name such as Fom0, Fom1, or Fom2.

Fusarium wilt on watermelons is caused by Fusarium oxysporum Esp. niveum. The disease was first found in watermelons in South Carolina and Georgia but later spread throughout the watermelon-growing regions of the world (Martyn, R. D. 1991 “Resistance to races 0, 1, and 2 of Fusarium Wilt of Watermelon in Citrullus sp. PI-296341-FR” HortSci. 26(4):429-432). Over the years, many watermelon cultivars resistant to Fusarium wilt Fon1) and Fon1 have been released from breeding programs starting with W. A. Orton in 1907, and leading to more contemporary diploid and triploid (seedless) “commercial” lines such as “Fiesta” (Syngenta), “Summer Flavor 790” (Abbott & Cobb), and “Afternoon Delight” (Dwayne Palmer) (Orton, Wash. 1907 “On methods of breeding for disease-resistance” Proc. Soc. Hort. Sci. 5:28; for a more complete table of commercial watermelon lines and their resistance to Fon1, please see “Midwest Vegetable Production Guide for Commercial Growers 2013” ID-56 pg 97). Other varieties have combined Fon 0 and 1 resistance with resistance to other diseases such as anthracnose leaf blight in “AU-Sweet Scarlet” (Norton J. D. et al., 1995 “AU-Sweet Scarlet′ Watermelon” Hort Sci. 30(2):393-394). However, as the prevalence of Fon2 Fusarium wilt has increased throughout the US east coast, many of these cultivars have succumbed to wilt. In addition to susceptibility in the fruit producing watermelons, fusarium wilt also negatively impacts flower production in “pollenizer plants” used in seedless watermelon pollination (Gunter et al., 2012 “Staminate Flower Production and Fusarium Wilt Reaction of Diploid Cultivars Used as Pollenizers for Triploid Watermelon” Hort Technology 22(5)).

Recently, Fusarium oxysporum Esp. niveum. Race 3 (Fon race 3) has been isolated and characterized (zhou et al., Race 3, a New and Highly Virulent Race of Fusarium oxysporum f. sp. niveum Causing Fusarium Wilt in Watermelon, Plant Disease/Vol. 94 No. 1), which is incorporated herein by reference in its entirety.

Fon2 is wide spread on the East coast of the US and becoming a growing problem with the introduction of Fon1 resistance. While Fon1 is currently the predominant commercial disease as resistance to Fon1 is introduced, Fon2 seems to move into the open niche and becomes a major issue. Chemical treatment is not available for Fusarium oxysporum disease. Fusarium resistant watermelon plants are also described in U.S. Pat. Nos. 7,550,652 and 8,212,116, U.S. Patent Application Publication No. 2010235941, and International Patent Publication Nos. WO2010098670 and WO2009000736, each of which is herein incorporated by reference in its entirety.

Any source of Fon2 resistance known to breeders can be used for the present invention. In some embodiments, the sources of Fon2 resistance include, but are not limited to, PI296341, PI482246, PI482252, PI296335, PI1271769, PI 255136, PI 270564, PI271769, USVL246-FR2, USVL252-FR2, USVL335-FR2, ‘AU-Sweet Scarlet’, among others (USDA Notice of release of watermelon germplasm lines USVL246-FR2 USVL252-FR2, and USVL335-FR2 Resistant to race 2 of Fusarium oxysporum f.sp. niveum; Boyhan, G. E. 2003 “Resistance to Fusarium Wilt and Root-knot; Wechter et al., 2012 “Identification of Resistance to Fusarium oxysporum f.sp. niveum Race 2 in Citrullus lanatus var. citroides Plant Introductions” HortSci. 47(3):334-338, Norton et al., HORTSCIENCE 30(2):393-394. 1995.).

### Watermelons

Watermelons are an economically important crop of the cucurbitaceae family comprising two subfamilies, eight tribes and 825 species (Jeffrey 1990 “An outline classification of the Cucurbitaceae” Biology and utilization of the Cucurbiticeae ed. D. M Bates 449-63 Ithaca N.Y.). In the US, cultivated watermelons varieties include C. lanatus var lanatus, C. lanatus var citroides, and C. colocynthis (Sheng, Yunyan 2012. “Genetic Diversity within Chinese Watermelon Ecotypes Compared with Germplasm from Other Countries” J. Amer. Soc. Hort. Sci 137(3):144-151).

TABLE 1
Selected commercial lines and breeding parentage.

| | | Breeding | | Fruit | | Flesh | |
|---|---|---|---|---|---|---|---|
| Cultivar | Source | parentage | Year | Shape | Wt | Color | Rind Color |
| AU-Jubilant | Hollar | Jubilee × PI 271778 | 1985 | Long | 25 | Light Red | Light green w/green |
| | | | | | | | narrow stripes |
| AU-Producer | Hollar | Crimson Sweet × PI | 1985 | Globe | 20 | Light red | Light green w/green |
| | | 189225 | | | | | wide stripes |
| Dixielee | Hollar | Texas W5, Wilt | 1979 | Globe | 20 | Deep red | Light green w/green |
| | | resistant Peacock, | | | | | narrow stripes |
| | | Fairfax, Summit | | | | | |
| Garrisonian | Willhite | Africa 8, Iowa Belle, | 1957 | Long | 20 | Light Red | Light green w/green |
| | | Garrison, | | | | | narrow stripes |
| | | Hawkesbury, | | | | | |
| | | Leesburg | | | | | |
| Charleston | NSL- | Africa 8, Iowa Belle | 1954 | Long | 20 | Light red | Light green/gray |
| Gray | 5267 and | Garrison, | | | | | |
| | NKL&G | Hawksbury, | | | | | |
| | | Leesburg | | | | | |

Table adapted from Levi and Thomas 2001 “Low Genetic Diversity Indicates the Need to Broaden the Genetic Base of Cultivated Watermelon” HortSci. 36(6): 1096-1101.

Watermelons have become an integral part of the American summer diet. According to the Agricultural Marketing Resource Center (AMRC), In 2005 Americans consumed an average of 13.8 pounds of watermelon fruit per person (Geishler 2007 “Watermelon” AMRC March, 2008). In order to meet consumer preferences, watermelon breeders have focused on producing a variety of watermelons with specific characteristics in the categories of yield, fruit shape, fruit size (weight), flesh color, seed content, and sweetness.

Watermelon, Citrullus lanatus (Thunb.) Matsum. & Nakai (2n=2x=22), belongs to the botanical family Cucurbitaceae. It is an important specialty crop accounting for 7% of the world area devoted to vegetable crops; and with annual worldwide production of ˜90 million tons (2000-2009). Over 83% of watermelons are produced in Asia with China being the leading producer, accounting for approx. 67% of the total world production. Same as many other cucurbit crops, knowledge and resources of watermelon genetics and genomics are currently very limited.

Linkage maps of watermelon crosses have described some QTLs associated with for agronomic traits of hardness of the rind, Brix of flesh juice, flesh color, and rind color among others (Hashizume T et al., 2003 Construction of a linkage map and QTL analysis of horticultural traits for watermelon [Citrullus lanatus (THUMB.) MASUM & NAKAI] using RAPD, RFLP and ISSR markers” Theor Appl Genet 106:779-785; Levi A, and Thomas C E 2006. “An Extended Linkage Map for Watermelon Based on SRAP, AFLP, SSR, ISSR, and RAPD Markers” J. Amer. Soc. Hort Sci. 131(3): 393-402; Sandlin et al., 2012 “Comparative mapping in watermelon [Citrullus lanatus (Thunb.) Masum. Et Nakai]” Theor Appl Genet 125:1603-1618; Levi et al., 2011 “An Extended Genetic Linkage Map for Watermelon Based on a Testcross and a BC₂F₂ Population” Am J of Plant Sci 2,93-110).

In addition, to accelerate watermelon breeding and understanding of its biology, the International Watermelon Genomics Initiative (IWGI) was formed in 2008 with one of its main goals being sequencing the whole genome of watermelon. The initiative is led by the National Engineering Research Center for Vegetables (NERCV), China and includes several other major participants: Beijing Genomics Institute (BGI-Shenzhen), Boyce Thompson Institute for Plant Research, National Research Institute of Agronomy (INRA) Center in Clermont-Ferrand (France), Institute of Vegetables and Flowers of Chinese Academy of Agricultural Sciences (IVF-CAAS), Xinjiang Academy of Agricultural Sciences, Syngenta seed company, and Ruk Zwaan seed company.

Watermelon has eleven chromosomes and a haploid genome of ˜425 Mb. Genome of domestic watermelon 97103 have been sequenced and assembled. A total of 46.18 Gb high-quality base pairs have been generated by Illumima Solexa Sequencing technology, which is about 107.4 fold coverage of the genome. The assembled N50 contig and scaffold sizes are 26,381 and 2,378,183 bp, respectively. 93.5% of the assembled sequence has been anchored onto the eleven chromosomes, among which ˜65% were oriented. A total of 23,440 genes were predicted in the current watermelon genome assembly (Cururbit Genomics Database, International Cucurbit Genomics Initiative (ICuGI)).

Methods for transforming watermelon have been described in Wang et al. (Genetic Transformation of Watermelon with Pumpkin DNA by Low Energy Ion Beam-Mediated Introduction, 2002 Plasma Sci. Technol. 4 1591), Zakaria et al. (Regeneration and Agrobacterium-Mediated Transformation of Watermelon, 2007, Pak. J. Biotechnol. Vol 4(1-2):15-23), Choi et al. (Genetic transformation and plant regeneration of watermelon using Agrobacterium tumefaciens, Plant Cell Reports, March 1994, Volume 13, Issue 6, pp 344-348), and Suratman et al. (Cotyledon with Hypocotyl Segment as an Explant for the Production of Transgenic Citrullus vulgaris Schrad (Watermelon) Mediated by Agrobacterium tumefaciens, Biotechnology 9 (2): 106-118, 2010), each of which is incorporated herein by reference in its entirety.

### Yield

Genetics of watermelon yield is described in Sidhu et al. (1977, Heterosis and combining ability of yield and its components in watermelon (Citrullus lanatus (Thunb.) Mansf.) Journal of Research 14:52-58; Mode of inheritance and gene action for yield and its components in watermelon (Citrullus lanatus (Thumb), Mansf). Journal of Research of Punjab Agriculture University 14:419-422).

### Shape

Watermelon fruit can be round, oval, blocky, or elongate in shape. The inheritance of fruit shape has not been widely studied, but the round, oval, and elongate phenotypes were shown to be determined by the incomplete dominance of the O gene. The homozygous dominant plants had elongated fruit, the homozygous recessive fruit were round (spherical), and the heterozygous fruit were oval (Weetman, L. M. 1937. Inheritance and correlation of shape, size and color in the watermelon, Citrullus vulgaris Schrad. Iowa Agricultural Experimental Station Annual Bulletin 228:224-256; Warid, A., and A. A. Abd el Hafez. 1976. Inheritance of marker genes of leaf color and ovary shape in watermelon. The Lybian Journal of Science 6:1-8.)

### Size (Weight)

The fruit of cultivated watermelon can vary in weight in size. Currently there are six recognized size categories of commercial watermelon: Giant (>14.5 kg), large (11.1-14.5 kg), medium (8.1-11.0 kg), small or pee-wee (5.5-8.0 kg), icebox (about 4.0 to 5.5 kg), and mini (less than 4.0 kg). Though watermelons of all sizes are available, recent years have seen a rise in the popularity of “small” watermelons as dessert for parties (Gusmini and Wehner, 2007 “Heritability and Genetic Variance Estimates for Fruit Weight in Watermelon”. Genetics of watermelon fruit weight is described in Sharma et al. (1988, Studies on some quantitative characters in watermelon (Citrullus lanatus Thunb. Mansf) I. Inheritance of earliness and fruit weight. Indian Journal of Horticulture 45:80-84).

Watermelons of different sizes may be obtained through various breeding methods. Currently there are no clear genes or QTLs for watermelon fruit weight. Instead, heredity is estimated by measuring the fruit size variance of several generations to estimate the broad and narrow-sense heritability of fruit size traits in various crosses. This application discusses many of the most common breeding techniques and methods for producing watermelon varieties of sizes via crosses with the Fon2 resistant plant of the present invention.

### Flesh

Watermelon flesh color is largely determined by its carotenoid content which in addition to creating different visual appearances, also defines fruit flavor via the production of several volatile aroma and flavor compounds (Lewinson E el al., 2005 “Carotenoid Pigementation Affects the Volatile Composition of Tomato and Watermelon Fruits, As Revealed by Comparative Genetic Analyses” J. Agric Food Chem 53, 3142-3148)

Watermelon fruits can come in a variety of colors including red, orange, salmon yellow, canary yellow, and white (Guner and Wehner 2003, “Gene list for watermelon”. Cucurbit Genet Coop Rep 26:76-92). The genetics in flesh color development are largely known and include three alleles identified as the y locus (Henderson 1989 “Inheritance of orange flesh color in watermelon” Cucurbit Genet Coop Rep 15:110; Henderson et al., 1998 “Interaction of flesh color genes in watermelon” J Hered 89:50-53; Poole 1944 “Genetics of cultivated cucurbits” J Hered 35:122-128; Porter 1937 “Inheritance of certain fruit and seed characters in watermelons” Hilgardia 10:489-509; Bang H et al., 2010 “Flesh Color Inheritance and Gene interactions among Canary Yellow, Pale Yellow, and Red Watermelon” J Amer. Soc. Hort. Sci. 135(4):362-368). Although breeders have access to genetic stocks for all of the flesh colors, consumer preference appears to be largely skewed towards red varieties (Evans 2008 “Consumer Preferences for Watermelons: a Conjoin Analysis” Auburn University Theses and Dissertations records), each of which is incorporated herein by reference in its entirety. All commercial red flesh varieties lack Fon2 resistance. For more background of genetic analyses of watermelon fruit pigmentation, see Lewinsohn et al. (J. Agric. Food Chem. 2005, 53, 3142-3148), Yoo et al. (Hort. Environ. Biotechnol. 53(6):552-560. 2012.), and Bang et al. (J. AMER. SOC. HORT. SCI. 135(4):362-368. 2010.)

Watermelon flesh firmness is another characteristics that breeders are interested in. Genetic locus controlling flesh firmness and methods of making watermelon plants with desired flesh firmness are described in U.S. Patent Application Publication No. 20130055466A1, which is incorporated by reference herein in its entirety.

### Seed Content

According to the National Watermelon Promotion Board 68% of the watermelons sold in the United States in 2003 were seedless (NWPB 2003 retail kit; and Evans 2008 “Consumer Preferences for Watermelons: a Conjoin Analysis” Auburn University Theses and Dissertations records). This trend has likely been growing as consumer preference continues to shift towards seedless watermelon.

Seedless watermelons are triploid hybrids produced by crossing diploid (2×) lines containing 22 chromosomes per cell with tetraploid (4×) lines containing 44 chromosomes per cell. This results in seeds that produce triploid (3×) plants with 33 chromosomes and are thus sterile “seedless fruits”. Methods for producing seedless watermelon are discussed in more detail later in this application.

### Polyploidy Watermelon

Polyploidy watermelon plants resistant to Font having desired flesh color are within the scope of the present invention. In some embodiments, the polyploidy watermelon is a triploid plant, a tetraploid plant, etc. In some embodiments, the plants is a allopolyploid plant.

Polyploids can be induced both naturally and artificially. It is generally accepted that natural polyploidy is very commons in plants, especially in angiosperms (30% to 70% of today's angiosperms are thought to be polyploids (Grant, B. 1971). Polyploidy naturally occurs in two ways: in some cases a somatic (non-reproductive) mutation may happen, due to interrupted mitosis, leading to chromosome doubling in a meristematic cells(s) that will produce a polyploid shoot; in other cases, polyploidy can be caused by the union of unreduced gametes (eggs and/or sperm that have not undergone normal meiosis and still have a 2n constitution).

There are several ways to artificially induce polyploidy, including but are not limited to, using environmental shock, applying chemicals, utilizing mutations that interrupt genome stability. In animals, hydrostatic pressure shock is used to polyploidize oysters (U.S. Pat. No. 4,834,024). Chemicals including adenine, noscapine hydrchloride, nocodazole, histone deacetylase inhibitor have been reported to induce polyploid animal cells (Edwards, A. et al 1999; Schuler, M. et al 1999; Verdoodt, B. et al 1999; Xu W. et al 2005). Increased expression of Clast3 gene can induce polyploid in human cells (Bahar, R. et al 2002).

Several chemicals and physical treatments are known to induce chromosome doubling in plant cells. For example, colchicine, nitrous oxide gas, heat treatment, amiprophos methyl, trifluralin, oryzalin, and pronamide have been used to obtain progenies with doubled chromosome number in many plant species. Those chemicals and physical treatments are also used for chromosome counting because these treatments arrest mitosis and accumulates mitotic figures in the specimens. Among these method, applying colchicine, a toxic alkaloid spindle inhibitor, extracted from the seeds of the autumn crocus (Colchicum autumnale), or colcemid, a synthetic equivalent, has been widely used as a chromosome doubling agent. Colchicine can dissociate the spindle and preventing migration of daughter chromosomes to opposite poles, resulting in polyploid tissue. It is normally applied to the meristematic regions of the plant (including germinating seeds, young seedlings and roots) by wetting with an aqueous solution, by spraying on in an emulsion, or by rubbing on in a lanolin paste. The effectiveness are affected by many factors, including the concentration of the solution, temperature and duration of the treatment, presence of adjuvant (e.g. dimethyl sulfoxide), depending on varied species and the part of the treatment. After the treatment, polyploid tissue can be identified and subjected to tissue culture step to generate polyploid plant (e.g. U.S. Pat. No. 6,747,191; U.S. application Ser. No. 10/573,340). Nitrous oxide gas (N₂O) is also used for chromosome doubling. The chromosome doubling effects of nitrous oxide gas were observed by Ostergren (1954) and have been used to induce chromosome doubling in wheat (Triticum dicoccum, Dvorak et al., 1973;), wheat haploids (Triticum aestivum L; Hansen et al., 1988), barley (Hordeum vulgare L., Dvorak et al., 1973), red clover (Trifolium pratense L., Taylor et al., 1976), oat (Avena saliva L., Dvorak and Harvey, 1973), Russian wildrye (Psathyrostachys juncea (Fisch.) Nevski, Berdahl and Barker, 1991), potato (Solanum tuberosum L.; Montezuma-de-Carvalho, 1967) and tulip (Tulipa spp., Zeilinga and Schouten, 1968). N₂O has been shown to induce partial chromosome doubling in maize shoot meristem and root tips (Kato, 1997). Polyploidy in plants can also be induced by heat treatment (Randolph, L. F. 1932).

Polyploidy is significant in plant breeding since it provides possibilities of greater expression of existing genetic diversity. The character of a plant can be changed by breeders through altering the number of genomes and consequently the dosage of allelic genes contributing to particular characters. Besides, polyploid plants may have adaptive and evolutionary advantages compared to diploids due to the higher degree of heterozygosity, especially for allopolyploids, a greater degree of heterozygosity can contribute to heterosis or hybrid vigor. Meanwhile, due to the genetic redundancy, extra copies of genes can be mutated resulting in new traits without compromising critical functions. Many polyploids also appear to be more self-fertile (except for plants with odd number of ploidy levels, e.g. 3×, 5×, 7× . . . et al), and inbreeding is less deleterious for allopolyploids due to their higher heterozygosity.

Development of seedless (or sterile) cultivars is of a great interest. There are number of methods available for developing sterile plants. However, one of the most rapid and cost effective approaches for inducing sterility in a plant is by creating polyploids with uneven (odd) chromosome sets. Polyploids with an uneven number of chromosome sets, such as triploid or pentaploid, are generally infertile and can be used to generate seedless plant cultivars. The uneven number of genomes prevents complete bivalent pairing and makes the formation of euploid gametes unlikely which turn prevents the production of seeds. In most cases the these plants function normally except for reproduction, specifically meiosis. One approach for creating triploid plants is to generate plants from endosperm tissue, which originates from the fusion of three haploid nuclei (one from male gametophyte and two from the female). This endosperm tissue can be isolated from developing seeds and cultured in vitro (tissue culture) to create a triploid plant. This approach has been successful for plants including citrus, kiwifruit, loquat, passionflower, acacia, rice, and pawpaw. In other cases, tetraploids can then be hybridized with diploids to create sterile triploids. For instance, triploid watermelons (U.S. Pat. Nos. 7,238,866; 7,164,059; 7,115,800) have been produced by using this method.

Polyploid can also enlarge and enhance hybrid vigor. Although enlarged cell size found in some polyploids can have undesirable effects, it can also be beneficial. In some plants, polyploidy results in significant enlargement and biomass increase (e.g. tetraploid apple fruit can be twice as large as diploid fruit). This type of enlargement is particularly desirable for ornamental flowers since flower petals can be thicker and longer lasting in polyploid plants (Kehr, 1996). Efforts have been made to artificially generate allopolyploid crop species. A widely used method is to cross two different species first and double the chromosomes to get a genetically fertile and stable hybrid polyploid plant which is diploid for two genomes derived from different species (also called amphidiploid). A good example is Triticale (U.S. Pat. No. 7,307,202). It is a crop species resulting from a synthetic species produced by crossing wheat (Triticum) and rye (Secale) and doubling the chromosome number. Triticale can provide grains with a milling quality approaching that of wheat on some soils primarily suitable for rye. It has been established as a valuable crop and more particularly where conditions are less favorable for wheat cultivation.

Non-limiting examples of methods for creating polyploidy watermelon are described in Chopra et al. (Induction of polyploidy in watermelon, Proceedings of the Indian Academy of Sciences—Section B, February 1960, Volume 51, Issue 2, pp 57-65), Gaikwad et al. (Induction of polyploidy in watermelon (Citrullus lanatus (Thunb.) Matsum and Nakai.)., Journal Agricultural & Biological Research 2009 Vol. 25 No. 2 pp. 110-118), Raza et al. (In Vitro Induction of Polyploids in Watermelon and Estimation Based on DNA Content, INTERNATIONAL JOURNAL OF AGRICULTURE & BIOLOGY 1560-8530/2003/05-3-298-302), Gunter et al. (HortTechnology, October 2012 22(5)), U.S. Pat. Nos. 6,759,576, 7,071,374, 7,238,866, 7,547,550, 7,652,193, 7,528,298, 8,476,500, 8,418,635, 8,418,637, 6,858,777 and Chinese Patent Application Publication CN1994065A, each of which is herein incorporated by reference in its entirety.

### Sweetness

One of the major factors affecting consumer choice of watermelons is taste and sweetness of the edible flesh. The accumulation of sugars in fruits is a consequence sugar translation and sugar biosynthesis. Watermelon fruit sweetness is largely determined by the presence of sucrose, fructose, and glucose sugars. The relative proportions of these sugars are regulated enzyme families of invertases, sucrose synthases, and sucrose phosphate synthases (Yativ M et al., 2010 “Sucrose accumulation in watermelon fruits: Genetic variation and biochemical analysis” J Plant Physiol 167(8) 589-96). The inheritance patterns of watermelon fruit sweetness are described in a study conducted by Yoo K. S. et al., (2012 Variation of Carotenoid, Sugar and Ascorbic Acid Concentrations in Watermelon Genotypes and Genetic Analysis” Hort. Environ. Biotechnol. 53(6):552-560).

Various measures are used to assess and describe different aspects of sweetness, but few are as popular as the measurement of soluble solid content (SSC, or Brix; Bumgarner and Matthew Kleinhenz 2012 “Using Brix as an indicator of Vegetable Quality: Instructions for measuring Brix in Cucumber, Leafy Greens, Sweet Corn, Tomato and Watermelon” H&CS department OSU HYG-1653-12).

Brix measurements can be conducted in a variety of ways including through the use of hydrometers in combination with Brix specific gravity tables. In other embodiments the sweetness of watermelons can be determined via techniques well known to those in the art including through spectral analysis using refractometers measuring the amount of light refracted from a liquid or with visible/near infrared diffuse transmittance techniques such as in U.S. Pat. No. 5,324,945 (Bumgarner and Matthew Kleinhenz 2012, OSU; and Hai-qing et al., 2007 “Measurement of soluble solids content in watermelon by Vis/NIR diffuse transmittance technique” J of Zhejiang Univ Sci B 8(2):105-110). Commercial watermelons of “breeder level” tend to have Brix sweetness values of greater than 3, greater than 4, greater than 5, greater than 6, greater than 7, greater than 8, greater than 9, greater than 10, greater than 11, greater than 12, greater than 13, greater than 14, greater than 15, greater than 16, greater than 17, greater than 18, greater than 19, greater than 20, greater than 20, greater than 21, or more. On the Brix scale for watermelons 7.8-8.2 is somewhat sweet, 8.3-9.0 is sweet, and >9.0 is very sweet. Below is a table with the Brix values of several commercial “breeder level” watermelon lines.

TABLE 2
Brix values of several commercial “breeder
level” watermelon lines

| | Variety | Brix | Days to Maturity |
|---|---|---|---|
| Personal/Mini | Belle 460 | 9.5 | 85 |
| Size | Betsy 8103 | 10 | 87 |
| | Cathay Belle | 10.2 | 82 |
| | Diana | 10.3 | 87 |
| | Gold Baby | 9.1 | 91 |
| | Gold Flower | 10.4 | 88 |
| | Golden Midget | 7.4 | 90 |
| Icebox Size | 7167 | 9.8 | 98 |
| | 7177 HQ | 11.4 | 89 |
| | 9651 HQ | 10.2 | 86 |
| | Afternoon Delight | 9.8 | 86 |
| | Amarillo | 10.4 | 89 |
| | Astrakhanski | 6.9 | 103 |
| | Crimson Tide | 8.8 | 91 |
| Picnic | 7187 HQ | 10.7 | 92 |
| | 9601 HQ | 10.2 | 87 |
| | Baby Doll | 8.9 | 89 |
| | Crimson Sweet | 9.9 | 89 |
| | Desert King | 8.6 | 95 |
| | Gypsy | 9.4 | 85 |
| | Harmony | 11.4 | 76 |

Table adapted from Washington State University Vegetable Research and Extension Program

Resistance to Fusarium oxysporum f. sp. niveum 2.

In 1991, a plant screening program at the Plant Protection Institute (Volcani Center-Israel), described the first and only available source of Fon2 resistance in a watermelon-PI-236341. This wild Citrullus line was originally collected from the Republic of South Africa by the Department of Agricultural Technical services, Pretoria and was deposited with the U.S. Plant Introduction Station in Beltsville, Md. in 1964. This line was to exhibit resistance to all three Fusarium oxysporum f. sp. niveum races, Fon0, 1, and 2 when inoculated at the 3-week stage (Martyn, R. D. and D. Netzer 1991. “Resistance to Races 0, 1, and 2 of Fusarium Wilt of Watermelon in Citrullus sp. PI-296341-FR” Hort. Science 26(4):429-432). Unfortunately, while the PI-236341 line could be bred to reliably inherit Fon2 resistance, its fruit was a incredibly small (usually 500 to 1200 g), and had several commercially undesirable traits such as grayish-green rind color, and a white, non-sweet flesh (considered bitter by some). Moreover, these negative fruit character traits (hard, white flesh, non-sweet) were discovered to be genetically linked to Fon2 resistance, making PI-296341 unusable for commercial breeding programs. To our knowledge, the present invention represents the first time the genetic linkage between Fon2 resistance and the undesirable fruit traits has been broken.

### Methods of Producing Plants Resistant to Fon2

Any watermelon plant raised from the deposited seeds that is resistant to Fon2 can be used to produce more watermelon plants that are resistant to Fon2 through plant breeding methods well known to those skilled in the art. The goal in general is to develop new, unique and superior varieties and hybrids. In some embodiments, selection methods, e.g., molecular marker assisted selection, can be combined with breeding methods to accelerate the process.

Choice of breeding or selection methods depends on the mode of plant reproduction, the heritability of the trait(s) being improved, and the type of cultivar used commercially (e.g., F1 hybrid cultivar, pure line cultivar, etc.). For highly heritable traits, a choice of superior individual plants evaluated at a single location will be effective, whereas for traits with low heritability, selection should be based on mean values obtained from replicated evaluations of families of related plants. Non-limiting breeding methods commonly include pedigree selection, modified pedigree selection, mass selection, recurrent selection, and backcross breeding.

The complexity of inheritance influences choice of the breeding method. Backcross breeding is used to transfer one or a few favorable genes for a heritable trait into a desirable cultivar. This approach has been used extensively for breeding disease-resistant cultivars, nevertheless, it is also suitable for the adjustment and selection of morphological characters, color characteristics and simply inherited quantitative characters. Various recurrent selection techniques are used to improve quantitatively inherited traits controlled by numerous genes. The use of recurrent selection in self-pollinating crops depends on the ease of pollination, the frequency of successful hybrids from each pollination and the number of hybrid offspring from each successful cross.

Each breeding program can include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.).

Promising advanced breeding lines are thoroughly tested per se and in hybrid combination and compared to appropriate standards in environments representative of the commercial target area(s) for three years at least. The best lines are candidates for use as parents in new commercial cultivars; those still deficient in a few traits may be used as parents to produce new populations for further selection.

In one embodiment, said method comprises (i) crossing any one of the Fon2 “breeder level” resistant plants of the present invention as a donor to a recipient plant line to create a F1 population. In one embodiment, the method further comprises (ii) evaluating Fon2 resistance in the offsprings derived from said F1 population. In one embodiment, the method further comprises (iii) selecting offsprings that are resistant to Fon2.

To select Fon2 resistant plants in the offsprings, a Fon2 resistant control plant and/or a Fon2 susceptible control plant can be involved. One such method for evaluation of Fon2 resistance involves inoculating the roots of offspring seedlings in Fon2 inocula for 10-14 seconds and returning them to soil growing conditions for later evaluation (Martyn, R. D. 1987 “Fusarium oxysporum f sp. nivcum race 2: A highly aggressive race new to the United States” Plant Dis. 71:233-236). In one embodiment, the evaluating step comprises visual observation to determine the percent wilting exhibited by each plant 1 week after inoculation (Martyn, R. D. 1987). By comparing wilting percentages across offspring and controls, the resistance level of offspring plants can be determined. In some embodiments, a screening and scoring system of the present invention, or any similar or substantially equivalent one can be used.

In another embodiment, said evaluating step comprises one or more molecular biological tests of pathogen density in the plants. In one embodiment, said molecular biological tests comprise testing the density of Fon2-specific nucleic acid sequence and/or Fon2-specific protein. For example, the molecular biological test can involve probe hybridization and/or amplification of nucleic acid (e.g., measuring viral nucleic acid density by Northern or Southern hybridization, RT-PCR) and/or immunological detection (e.g., measuring viral protein density, such as precipitation and agglutination tests, ELISA (e.g., Lateral Flow test or DAS-ELISA), Western blot, RIA, immunogold labeling, immunosorbent electron microscopy (ISEM), and/or dot blot). For example, a plant may be resistant to a Fon2 if it has a Fon2 nucleic acid and/or protein density that is about 50%, about 40%, about 30%, about 20%, about 10%, about 5%, about 2%, about 1%, about 0.1%, about 0.01%, about 0.001%, or about 0.0001% of the Fon2 nucleic acid and/or protein density in a susceptible plant.

The procedure to perform a nucleic acid hybridization, an amplification of nucleic acid (e.g., RT-PCR) or an immunological detection (e.g., precipitation and agglutination tests, ELISA (e.g., Lateral Flow test or DAS-ELISA), Western blot, RIA, immunogold labeling, immunosorbent electron microscopy (ISEM), and/or dot blot tests) are performed as described elsewhere herein and well-known by one skilled in the art.

In one embodiment, the evaluating step comprises RT-PCR (semi-quantitative or quantitative), wherein Fon2-specific primers are used to amplify one or more Fon2-specific nucleic acid sequences. In one embodiment, said Fon2-specific nucleic acid sequences are from the same gene of Fon2. In another embodiment, said Fon2-specific nucleic acid sequences are from different genes of Fon2. In one embodiment, said RT-PCR is a real-time RT-PCR.

In another embodiment, the evaluating step comprises immunological detection (e.g., precipitation and agglutination tests, ELISA (e.g., Lateral Flow test or DAS-ELISA), Western blot, RIA, immunogold labeling, immunosorbent electron microscopy (ISEM), and/or dot blot), wherein one or more Fon2-specific antibodies are used to detect one or more FON2-specific proteins. In one embodiment, said Fon2-specific antibody is selected from the group consisting of polyclonal antibodies, monoclonal antibodies, and combination thereof. In one embodiment, said Fon2-specific protein is a Fon2 cell wall protein.

Reverse Transcription Polymerase Chain Reaction (RT-PCR) can be utilized in the present invention to determine the fungal growth in a plant. It is a variant of polymerase chain reaction (PCR), a laboratory technique commonly used in molecular biology to generate many copies of a DNA sequence, a process termed “amplification”. In RT-PCR, however, RNA strand is first reverse transcribed into its DNA complement (complementary DNA, or cDNA) using the enzyme reverse transcriptase, and the resulting cDNA is amplified using traditional or real-time PCR.

RT-PCR utilizes a pair of primers, which are complementary to a defined sequence on each of the two strands of the cDNA. These primers are then extended by a DNA polymerase and a copy of the strand is made after each cycle, leading to logarithmic amplification.

RT-PCR includes three major steps. The first step is the reverse transcription (RT) where RNA is reverse transcribed to cDNA using a reverse transcriptase and primers. This step is very important in order to allow the performance of PCR since DNA polymerase can act only on DNA templates. The RT step can be performed either in the same tube with PCR (one-step PCR) or in a separate one (two-step PCR) using a temperature between 40° C. and 50° C., depending on the properties of the reverse transcriptase used.

The next step involves the denaturation of the dsDNA at 95° C., so that the two strands separate and the primers can bind again at lower temperatures and begin a new chain reaction. Then, the temperature is decreased until it reaches the annealing temperature which can vary depending on the set of primers used, their concentration, the probe and its concentration (if used), and the cations concentration. The main consideration, of course, when choosing the optimal annealing temperature is the melting temperature (Tm) of the primers and probes (if used). The annealing temperature chosen for a PCR depends directly on length and composition of the primers. This is the result of the difference of hydrogen bonds between A-T (2 bonds) and G-C (3 bonds). An annealing temperature about 5 degrees below the lowest Tm of the pair of primers is usually used.

The final step of PCR amplification is the DNA extension from the primers which is done by the thermostable Taq DNA polymerase usually at 72° C., which is the optimal temperature for the polymerase to work. The length of the incubation at each temperature, the temperature alterations and the number of cycles are controlled by a programmable thermal cycler. The analysis of the PCR products depends on the type of PCR applied. If a conventional PCR is used, the PCR product is detected using agarose gel electrophoresis and ethidium bromide (or other nucleic acid staining).

Conventional RT-PCR is a time-consuming technique with important limitations when compared to real time PCR techniques. This, combined with the fact that ethidium bromide has low sensitivity, yields results that are not always reliable. Moreover, there is an increased cross-contamination risk of the samples since detection of the PCR product requires the post-amplification processing of the samples. Furthermore, the specificity of the assay is mainly determined by the primers, which can give false-positive results. However, the most important issue concerning conventional RT-PCR is the fact that it is a semi or even a low quantitative technique, where the amplicon can be visualized only after the amplification ends.

Real time RT-PCR provides a method where the amplicons can be visualized as the amplification progresses using a fluorescent reporter molecule. There are three major kinds of fluorescent reporters used in real time RT-PCR, general non specific DNA Binding Dyes such as SYBR Green I, TaqMan Probes and Molecular Beacons (including Scorpions).

The real time PCR thermal cycler has a fluorescence detection threshold, below which it cannot discriminate the difference between amplification generated signal and background noise. On the other hand, the fluorescence increases as the amplification progresses and the instrument performs data acquisition during the annealing step of each cycle. The number of amplicons will reach the detection baseline after a specific cycle, which depends on the initial concentration of the target DNA sequence. The cycle at which the instrument can discriminate the amplification generated fluorescence from the background noise is called the threshold cycle (Ct). The higher is the initial DNA concentration, the lower its Ct will be.

In one embodiment, complete chromosomes of the donor plant are transferred. For example, the Fon2 resistant plant can serve as a male or female parent in a cross pollination to produce resistant offspring plants, wherein by receiving the genomic material from the resistant donor plant, the offspring plants are resistant to Fon2. Alternatively, a resistant plant can be cloned or produced via tissue culture so as to produce additional plants with resistance.

In another method for producing a Fon2 resistant plant, protoplast fusion can also be used for the transfer of resistance-conferring genomic material from a donor plant to a recipient plant. Protoplast fusion is an induced or spontaneous union, such as a somatic hybridization, between two or more protoplasts (cells of which the cell walls are removed by enzymatic treatment) to produce a single bi- or multi-nucleate cell. The fused cell that may even be obtained with plant species that cannot be interbred in nature is tissue cultured into a hybrid plant exhibiting the desirable combination of traits. More specifically, a first protoplast can be obtained from a plant line that is resistant to Fon2. For example, a protoplast from a Fon2-resistant (melon, watermelon, squash or cucumber) line may be used. A second protoplast can be obtained from a susceptible second plant line, optionally from another plant species or variety, preferably from the same plant species or variety that comprises commercially desirable characteristics, such as, but not limited to disease resistance, insect resistance, valuable fruit characteristics, etc. The protoplasts are then fused using traditional protoplast fusion procedures, which are known in the art to produce the cross.

Alternatively, embryo rescue may be employed in the transfer of resistance-conferring genomic material from a donor plant to a recipient plant. Embryo rescue can be used as a procedure to isolate embryo's from crosses wherein plants fail to produce viable seed. In this process, the fertilized ovary or immature seed of a plant is tissue cultured to create new plants (see Pierik, 1999, In vitro culture of higher plants, Springer, ISBN 079235267x, 9780792352679, which is incorporated herein by reference in its entirety).

As described before, the recipient line can be an elite line having certain favorite traits. In one embodiment, the elite line is resistant to Fon2 due to a different genetic cause other than slc-2 gene. When crossed together, different loci may provide quantitatively additive effect in terms of resistance to Fon2. In that case, QTL mapping can be involved to facilitate the breeding process.

A QTL (quantitative trait locus) mapping can be applied to determine the parts of the donor plant's genome conferring the Fon2 resistance, and facilitate the breeding methods. Inheritance of quantitative traits or polygenic inheritance refers to the inheritance of a phenotypic characteristic that varies in degree and can be attributed to the interactions between two or more genes and their environment. Though not necessarily genes themselves, quantitative trait loci (QTLs) are stretches of DNA that are closely linked to the genes that underlie the trait in question. QTLs can be molecularly identified to help map regions of the genome that contain genes involved in specifying a quantitative trait. This can be an early step in identifying and sequencing these genes.

Typically, QTLs underlie continuous traits (those traits that vary continuously, e.g. level of resistance to virus) as opposed to discrete traits (traits that have two or several character values, e.g. smooth vs. wrinkled peas used by Mendel in his experiments). Moreover, a single phenotypic trait is usually determined by many genes. Consequently, many QTLs are associated with a single trait.

A quantitative trait locus (QTL) is a region of DNA that is associated with a particular phenotypic trait—these QTLs are often found on different chromosomes. Knowing the number of QTLs that explains variation in the phenotypic trait tells about the genetic architecture of a trait. It may tell that plant resistance to virus of the present invention is controlled by many genes of small effect, or by a few genes of large effect.

Another use of QTLs is to identify candidate genes underlying a trait. Once a region of DNA is identified as contributing to a phenotype, it can be sequenced. The DNA sequence of any genes in this region can then be compared to a database of DNA for genes whose function is already known.

In a recent development, classical QTL analyses are combined with gene expression profiling i.e. by DNA microarrays. Such expression QTLs (e-QTLs) describes cis- and trans-controlling elements for the expression of often disease-associated genes. Observed epistatic effects have been found beneficial to identify the gene responsible by a cross-validation of genes within the interacting loci with metabolic pathway- and scientific literature databases.

QTL mapping is the statistical study of the alleles that occur in a locus and the phenotypes (physical forms or traits) that they produce (see, Meksem and Kahl, The handbook of plant genome mapping: genetic and physical mapping, 2005, Wiley-VCH, ISBN 3527311165, 9783527311163). Because most traits of interest are governed by more than one gene, defining and studying the entire locus of genes related to a trait gives hope of understanding what effect the genotype of an individual might have in the real world.

Statistical analysis is required to demonstrate that different genes interact with one another and to determine whether they produce a significant effect on the phenotype. QTLs identify a particular region of the genome as containing a gene that is associated with the trait being assayed or measured. They are shown as intervals across a chromosome, where the probability of association is plotted for each marker used in the mapping experiment.

To begin, a set of genetic markers must be developed for the species in question. A marker is an identifiable region of variable DNA. Biologists are interested in understanding the genetic basis of phenotypes (physical traits). The aim is to find a marker that is significantly more likely to co-occur with the trait than expected by chance, that is, a marker that has a statistical association with the trait. Ideally, they would be able to find the specific gene or genes in question, but this is a long and difficult undertaking. Instead, they can more readily find regions of DNA that are very close to the genes in question. When a QTL is found, it is often not the actual gene underlying the phenotypic trait, but rather a region of DNA that is closely linked with the gene.

For organisms whose genomes are known, one might now try to exclude genes in the identified region whose function is known with some certainty not to be connected with the trait in question. If the genome is not available, it may be an option to sequence the identified region and determine the putative functions of genes by their similarity to genes with known function, usually in other genomes. This can be done using BLAST, an online tool that allows users to enter a primary sequence and search for similar sequences within the BLAST database of genes from various organisms.

Another interest of statistical geneticists using QTL mapping is to determine the complexity of the genetic architecture underlying a phenotypic trait. For example, they may be interested in knowing whether a phenotype is shaped by many independent loci, or by a few loci, and do those loci interact. This can provide information on how the phenotype may be evolving.

Molecular markers are used for the visualization of differences in nucleic acid sequences. This visualization is possible due to DNA-DNA hybridization techniques (RFLP) and/or due to techniques using the polymerase chain reaction (e.g. STS, microsatellites, AFLP). All differences between two parental genotypes will segregate in a mapping population based on the cross of these parental genotypes. The segregation of the different markers may be compared and recombination frequencies can be calculated. The recombination frequencies of molecular markers on different chromosomes are generally 50%. Between molecular markers located on the same chromosome the recombination frequency depends on the distance between the markers. A low recombination frequency corresponds to a low distance between markers on a chromosome. Comparing all recombination frequencies will result in the most logical order of the molecular markers on the chromosomes. This most logical order can be depicted in a linkage map (Paterson, 1996). A group of adjacent or contiguous markers on the linkage map that is associated to a reduced disease incidence and/or a reduced lesion growth rate pinpoints the position of a QTL.

The nucleic acid sequence of a QTL may be determined by methods known to the skilled person. For instance, a nucleic acid sequence comprising said QTL or a resistance-conferring part thereof may be isolated from a Fon2-resistant donor plant by fragmenting the genome of said plant and selecting those fragments harboring one or more markers indicative of said QTL. Subsequently, or alternatively, the marker sequences (or parts thereof) indicative of said QTL may be used as (PCR) amplification primers, in order to amplify a nucleic acid sequence comprising said QTL from a genomic nucleic acid sample or a genome fragment obtained from said plant. The amplified sequence may then be purified in order to obtain the isolated QTL. The nucleotide sequence of the QTL, and/or of any additional markers comprised therein, may then be obtained by standard sequencing methods.

One or more such QTLs associated with the resistance to Fon2 in a donor plant can be transferred to a recipient plant that is susceptible to Fon2 to make it become resistant through any transferring and/or breeding methods.

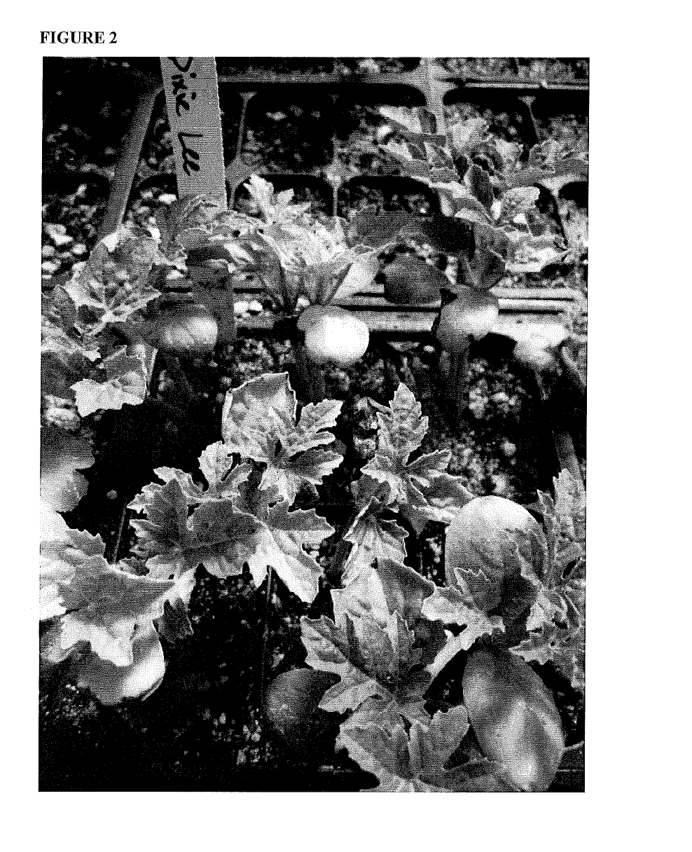

In one embodiment, an advanced backcross QTL analysis (AB-QTL) is used to discover the nucleotide sequence or the QTLs responsible for the resistance of a plant. Such method was proposed by Tanksley and Nelson in 1996 (Tanksley and Nelson, 1996, Advanced backcross QTL analysis: a method for simultaneous discovery and transfer of valuable QTL from un-adapted germplasm into elite breeding lines. Theor Appl Genet 92:191-203) as a new breeding method that integrates the process of QTL discovery with variety development, by simultaneously identifying and transferring useful QTL alleles from un-adapted (e.g., land races, wild species) to elite germplasm, thus broadening the genetic diversity available for breeding. A non-limiting exemplary scheme of AB-QTL mapping strategy is shown in FIG. 2. AB-QTL strategy was initially developed and tested in tomato, and has been adapted for use in other crops including rice, maize, wheat, pepper, barley, and bean. Once favorable QTL alleles are detected, only a few additional marker-assisted generations are required to generate near isogenic lines (NILS) or introgression lines (ILs) that can be field tested in order to confirm the QTL effect and subsequently used for variety development.

Isogenic lines in which favorable QTL alleles have been fixed can be generated by systematic backcrossing and introgressing of marker-defined donor segments in the recurrent parent background. These isogenic lines are referred as near isogenic lines (NILs), introgression lines (ILs), backcross inbred lines (BILs), backcross recombinant inbred lines (BCRIL), recombinant chromosome substitution lines (RCSLs), chromosome segment substitution lines (CSSLs), and stepped aligned inbred recombinant strains (STAIRSs). An introgression line in plant molecular biology is a line of a crop species that contains genetic material derived from a similar species. ILs represent NILs with relatively large average introgression length, while BILs and BCRILs are backcross populations generally containing multiple donor introgrcssions per line. As used herein, the term “introgression lines or ILs” refers to plant lines containing a single marker defined homozygous donor segment, and the term “pre-ILs” refers to lines which still contain multiple homozygous and/or heterozygous donor segments.

To enhance the rate of progress of introgression breeding, a genetic infrastructure of exotic libraries can be developed. Such an exotic library comprises of a set of introgression lines, each of which has a single, possibly homozygous, marker-defined chromosomal segment that originates from a donor exotic parent, in an otherwise homogenous elite genetic background, so that the entire donor genuine would be represented in a set of introgression lines. A collection of such introgression lines is referred as libraries of introgression lines or IL libraries (ILLs). The lines of an ILL cover usually the complete genome of the donor, or the part of interest. Introgression lines allow the study of quantitative trait loci, but also the creation of new varieties by introducing exotic traits. High resolution mapping of QTL using ILLs enable breeders to assess whether the effect on the phenotype is due to a single QTL or to several tightly linked QTL affecting the same trait. In addition, sub-ILs can be developed to discover molecular markers which are more tightly linked to the QTL of interest, which can be used for marker-assisted breeding (MAB). Multiple introgression lines can be developed when the introgression of a single QTL is not sufficient to result in a substantial improvement in agriculturally important traits (Gur and Zamir, Unused natural variation can lift yield barriers in plant breeding, 2004, PLoS Biol.; 2(10):e245).

### Breeding Methods

Open-Pollinated Populations. The improvement of open-pollinated populations of such crops as rye, many maizes and sugar beets, herbage grasses, legumes such as alfalfa and clover, and tropical tree crops such as cacao, coconuts, oil palm and some rubber, depends essentially upon changing gene-frequencies towards fixation of favorable alleles while maintaining a high (but far from maximal) degree of heterozygosity. Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes for flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

There are basically two primary methods of open-pollinated population improvement. First, there is the situation in which a population is changed en masse by a chosen selection procedure. The outcome is an improved population that is indefinitely propagable by random-mating within itself in isolation. Second, the synthetic variety attains the same end result as population improvement but is not itself propagable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988).

Mass Selection.

In mass selection, desirable individual plants are chosen, harvested, and the seed composited without progeny testing to produce the following generation. Since selection is based on the maternal parent only, and there is no control over pollination, mass selection amounts to a form of random mating with selection. As stated above, the purpose of mass selection is to increase the proportion of superior genotypes in the population.

Synthetics.

A synthetic variety is produced by crossing inter se a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or toperosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous.

Whether a synthetic can go straight from the parental seed production plot to the farmer or must first undergo one or two cycles of multiplication depends on seed production and the scale of demand for seed. In practice, grasses and clovers are generally multiplied once or twice and are thus considerably removed from the original synthetic.

While mass selection is sometimes used, progeny testing is generally preferred for polycrosses, because of their operational simplicity and obvious relevance to the objective, namely exploitation of general combining ability in a synthetic.

The number of parental lines or clones that enters a synthetic varies widely. In practice, numbers of parental lines range from 10 to several hundred, with 100-200 being the average. Broad based synthetics formed from 100 or more clones would be expected to be more stable during seed multiplication than narrow based synthetics.

Hybrids.

A hybrid is an individual plant resulting from a cross between parents of differing genotypes. Commercial hybrids are now used extensively in many crops, including corn (maize), sorghum, sugarbeet, sunflower and broccoli. Hybrids can be formed in a number of different ways, including by crossing two parents directly (single cross hybrids), by crossing a single cross hybrid with another parent (three-way or triple cross hybrids), or by crossing two different hybrids (four-way or double cross hybrids).

Strictly speaking, most individuals in an out breeding (i.e., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

The production of hybrids is a well-developed industry, involving the isolated production of both the parental lines and the hybrids which result from crossing those lines. For a detailed discussion of the hybrid production process, see, e.g., Wright, Commercial Hybrid Seed Production 8:161-176, In Hybridization of Crop Plants.

Bulk Segregation Analysis (BSA). BSA, a.k.a. bulked segregation analysis, or bulk segregant analysis, is a method described by Michelmore et al. (Michelmore et al., 1991, Identification of markers linked to disease-resistance genes by bulked segregant analysis: a rapid method to detect markers in specific genomic regions by using segregating populations. Proceedings of the National Academy of Sciences, USA, 99:9828-9832) and Quarrie et al. (Quarrie et al., Bulk segregant analysis with molecular markers and its use for improving drought resistance in maize, 1999, Journal of Experimental Botany, 50(337):1299-1306).

For BSA of a trait of interest, parental lines with certain different phenotypes are chosen and crossed to generate F2, doubled haploid or recombinant inbred populations with QTL analysis. The population is then phenotyped to identify individual plants or lines having high or low expression of the trait. Two DNA bulks are prepared, one from the individuals having one phenotype (e.g., resistant to virus), and the other from the individuals having reversed phenotype (e.g., susceptible to virus), and analyzed for allele frequency with molecular markers. Only a few individuals are required in each bulk (e.g., 10 plants each) if the markers are dominant (e.g., RAPDs). More individuals are needed when markers are co-dominant (e.g., RFLPs). Markers linked to the phenotype can be identified and used for breeding or QTL mapping. In one embodiment, Fon2 “breeder level” resistant plants of the present invention are crossed to produce triploid “seedless” watermelons. Triploid seeds are produced by crossing diploid (2×) lines containing 22 chromosomes per cell with tetraploid (4×) lines containing 44 chromosomes per cell. This results in seeds that produce triploid (3×) plants with 33 chromosomes. Triploid plants are true F.sub.1 hybrids so their production depends on development of diploid and tetraploid parental lines (Wall [1960] Am. Soc. Hort. Sci. 76:577-588).

For large-scale commercial production of triploid seed, tetraploid and diploid parental lines are planted in mixed plots and allowed to cross pollinate. Triploid seed is produced only in melons on tetraploid plants that are fertilized with diploid pollen. Therefore, an adequate supply of diploid and tetraploid seed must be available to produce large mixed stands. All commercially grown seeded watermelons are diploid; therefore, lines for use as diploid parents are abundant. The major limitation to producing seedless watermelon lies in the difficulty associated with producing sufficient seed for the tetraploid (4×) parental lines which are eventually pollinated with a diploid (2×) to produce the seedless triploid (3×) seed.

Tetraploid lines are produced from diploid seedlings by application of colchicine. With either diploid or tetraploids, once a desirable cultivar is identified, the plant is self-pollinated in order to build up adequate seed. Diploid seed is easily produced by open pollination of pure stands of a given diploid cultivar. Tetraploid seed, however, has proven to be very difficult to produce in large, commercially useful, quantities. This is largely due to the fact that tetraploids exhibit a high degree of self-sterility. As a result of this self-sterility, very few melons develop in a field of tetraploid plants. Also, none or only a small number of seeds are usually produced in each self-pollinated melon.

Two commonly used methods to cross tetraploid (4×) and diploid (2×) are described here. These methods are not meant to be an exhaustive list as variations to these methods can be made according to actual production situation and with other advancements in the field such as for examples those described in U.S. Pat. Nos. 5,007,198 and 8,418,637 or with watermelon varieties designed for these pollination crosses such as those described in U.S. Pat. No. 6,759,576, and 7,071,374.

### Hand-Pollination Method

Wherein the inbred tetraploid female parent and the inbred diploid male parent line are planted in the same field. The inbred male parent is planted 7-10 days earlier than the female parent to insure adequate pollen supply at the pollination time. The male parent and female parent are planted in the ratio of 1 male parent to 4-10 female parents. The diploid male parent may be planted at the top of the field for efficient male flower collection during pollination. Pollination is started when the second female flower on the tetraploid female parent is ready to flower. Female flower buds that are ready to open the next day are identified, covered with paper cups or small paper bags that prevent bee or any other insect visit of the female flowers, and marked with any kind of material that can be easily seen the next morning. This process is best done in the afternoon. The male flowers of the diploid male parent are collected in the early morning before they are open and visited by pollinating insects. The covered female flowers of the tetraploid female parent, which have opened, are un-covered and pollinated with the collected fresh male flowers of the diploid male parent, starting as soon as the male flower sheds pollen. The pollinated female flowers are again covered after pollination to prevent bees and any other insects visit. The pollinated female flowers are also marked. Only the marked fruits are harvested for extracting triploid hybrid seed.

### Bee-Pollination Method

Using the bee-pollination method, the tetraploid female parent and the diploid male parent are usually planted in a ratio of 2 rows tetraploid parent to 1 row male parent. The female tetraploid plants are pruned to 2-3 branches. All the male flower buds on the female tetraploid parent plants are removed manually, (the de-budding process), during the pollination season on a daily basis. Beehives are placed in the field for transfer of pollen by bees from the male parent to the female flowers of the female parent. Fruits set during this de-budding time are marked. Only the marked fruits are harvested for extracting hybrid triploid seed.

Oftentimes, the pollinating watermelon plant is a specially bred “pollenizer” plant bred for its production of flowers for pollination of 4n female parents without regard to fruit quality. These pollenizer parents tend to have compact growth habits that offer less competition against the triploid cultivars. Some popular nonharvested pollenizer lines include ACX 9825 (Abbot and Cobb), Side Kick (Harris Moran), Jenny (Nunhems) 5WDL 6132 (Syngenta) among others (Gunter et al., 2012 “Staminate Flower Production and Fusarium Wilt Reaction of Diploid Cultivars Used as Pollenizers for Triploid Watermelon” Hort Technology 22(5); and U.S. Pat. No. 6,759,576 and U.S. Pat. No. 6,355,865). In some embodiments the watermelon plant of the present invention can be used as a pollenizer or can be used in a breeding program to produce a pollenizer variety.

This invention is further illustrated by the following examples. It should be understood that the examples and embodiments described herein are for illustrative purposes only and that various modifications or changes in light thereof will be suggested to persons skilled in the art and are to be included within the spirit and purview of this application.

### EXAMPLES

### Example 1

Watermelon consumers the world over prefer sweet, firm, crisp, but soft, red flesh watermelons. While some commercial cultivars with colored flesh exist (yellow, gold, swirl yellow and red), red flesh varieties command 85%+ market share.

Watermelon cultivars resistant to certain races of Fusarium wilt (Fon) have been released from many breeding programs, beginning with W. A. Orton (Orton, 1907). Many of these, however, have succumbed to wilt over the years due to the pathogenic variability (different races or strains) of the fungus.

At present there are three described races of Fusarium oxysporum f. sp. niveum: races 0, 1 and 2 (Cirulli 1972, Martyn 1987, Martyn and Netzer 1991). The most recently described is race 2 (Fon 2), first observed in Israel in 1973 (Netzer, 1976, Netzer and Dishon, 1973) and in the United States in 1981 (Martyn, 1985, 1987) (Martyn and Netzer, 1991). Recent reports have started to also suggest the existence of a 3^{rd} Fon race (Zhou X. G. 2010 “Race3, a New and Highly Virulent Race of Fusarium oxysporum Esp. niveum Causing Fusarium Wilt in Watermelon” Plant Disease Vol 94, pg 92-98; Martyn R. D. 2007 “Fusarium wilt of watermelon and other cucurbits” The Plant Health Instructor DOI: 10.1094/PHI-I-2007-0122-01) Race 2 is now present in Florida and Oklahoma (Martyn and Bruton, 1989). Many cultivars have high wilt resistance to isolates of races 0 and 1; however, race 2 is more aggressive and overcomes all currently known wilt-resistant cultivars.

Fusarium 2 (Fon 2) is a published resistance from a wild type relative (PI 296341-FR) with white flesh color and poor fruit quality. Negative linkage association (white flesh with Fon 2 resistance) has prohibited incorporation of Fon2 in commercial red flesh varieties.

Origin: PI-296341 (Citrullus sp.) was collected from the Republic of South Africa under the local name of Tsamma′ by the Dept. of Agricultural Technical Services, Pretoria, and was deposited with the U.S. Plant Introduction Station in Beltsville, Md. in 1964. (Martyn and Netzer, 1991).

PI-296341-FR was described in Martyn and Netzer, 1991. It produces small round grayish-green fruit with numerous small, olive-green to brown seeds. Approximate days to maturity are 80. The flesh is white and lacks a noticeable flavor but it is not bitter.

When small, the fruit is pubescent but loses that trait after several weeks. The vine is small-leaved compared to cultivated types and has numerous runners. This PI could be a potential source of resistance to Fon 2 in breeding programs (Martyn and Netzer, 1991).

We received the seed of the accession PI-296341-FR from Dan Egal (Purdue University). Dr. Egal received this accession from Ray Martyn who did a public release as PI-296341-FR (See Martyn, R. D. and Netzer, D., 1991).

This resistance is described as a monogenic and recessive. When attempting to incorporate the resistance into existing lines, the fruit of the survivors always looked like the PI with hard white flesh and low sugars. This fruit type is not marketable. Therefore, what is needed is Fon 2 resistance in elite watermelon breeding lines and commercial parents. These elite lines all require red flesh. Early work with this PI only produced white flesh off-spring. The genetics of flesh color are complicated but, in general, white is dominant. Also complicating the problem, the 2 subspecies (Citrullus lanatus lanatus (the elite line) and Citrullus lanatus citroides (PI296341-FR) have skewed segregation (the chromosomes preferentially pair within the subspecies.

Fon 1 is a typical soil pathogen and is endemic in areas where watermelons are grown. It can be seed transmitted. We estimate about 20%+ of fields have Fon 2 present. In Commercial production, Fon 1 resistant varieties seems to “open the niche” for the Fon 2 pathogen to attack commercial field causing economic losses. Chemical treatment is not available for Fusarium disease. At this time there is no known treatment for an infected field except to rotate out of watermelon for 7+ years.

### Technical Problem to be Solved

Negative linkage association composed of a negative fruit character (hard, white flesh) and Fon 2 resistance must be broken. Specifically, hard, white flesh with low sugar must be replaced with red flesh of acceptable market quality and Fon 2 resistance.

Linkage association seems to be confirmed in all of our initial trials because all Fon 2 resistant material that was bred from the PI consistently had white fleshed and hard, white flesh is a characteristic of the PI. Hard and white do seem to be inherited together, but this could be due to the skewed segregation. There is a soft, white heirloom open pollinated variety.

### The Technical Solution Applied

The solution applied was to break the linkage between the Fusarium 2 resistance gene(s) and the white flesh: this recessive gene was backcrossed into a recurrent parent and the progeny were selfed after each backcross. These selfed lines were then challenged with the disease Fon 2.

Survivors were screened with 50 neutral markers for the first marker assisted backcross. This allowed identification of individuals scoring as “more domesticated”. The markers used were neutral (not linked to a known trait) that were polymorphic between the 2 parents. There is no physical map of watermelon.

Methods of using neutral markers for plant breeding are described in Tanksley et al. (Nature Biotechnology, 7:257-264, March 1989); Hospital et al. (Genetics, 132:1199-1210, December 1992); Frisch et al., (Crop Sci. 39:967-975, 1999), Visscher et al. (Genetics, 144:1923-1932, December 1996), Kang (Quantitative Genetics, Genomics and Plant Breeding, CABI Publishing), and Randhawa et al. (PLoS ONE 4(6): e5752, June 2009), each of which is herein incorporated by reference in its entirety for all purposes. As work progressed through the backcross cycles, fewer markers were used since we were slowly getting rid of those associated with the PI. Over 6500 plants were screened during the course of this work. Only plants rated as resistant during the pathogen screen were tested with the markers, then the most ‘domestic” i.e. related to the elite parent were saved. These selections were then back-crossed to the recurrent parent, selfed, disease screened and selected again with markers. This cycle was repeated at least four times to get rid of the linkage drag and the other PI traits.

Standard Fon 2 disease screen protocol as described herein was used. 50 neutral markers used to identify less wild progeny. Field evaluation of fruit color and quality (texture, flavor, freedom from “off” tastes) was conducted.

Table 2 below is a description of the plants involved in the breeding process.

TABLE 2a
Breeding Pedigrees

| generation | pedigree | description | Selection | markers | eval |
|---|---|---|---|---|---|
| S3 | PI296341-FR | PI | | 54 | |
| F6 | SuMdgA2 [(Sultan treated with colchicine- selfed&sel 8 times)/(Mardi | P2 (recurrent) | | 54 | |
| | Gras-selfed&sel8 times)] | | | | |
| F1 | PI296341-FR/SuMdgA2 | P1 × P2 | | | |
| F2 | PI296341-FR/SuMdgA2 | F1 selfed | Fus2 screen | 54 | color |
| BC1 | (PI296341-FR/SuMdgA2)/SuMdgA2 | F2 × SuMdgA2 | | | |
| BC1S1 | (PI296341-FR/SuMdgA2)/SuMdgA2 | BC1 selfed | Fus2 screen | 37 | |
| S1BC2 | [(PI296341-FR/SuMdgA2)/SuMdgA2]/SuMdgA2 | BC1S1 BC | | | color |
| S1BC2S1 | [(PI296341-FR/SuMdgA2)/SuMdgA2]/SuMdgA2 | S1BC2 selfed | Fus2 screen | 48 | color |
| S2BC3 | [[(PI296341-FR/SuMdgA2)/SuMdgA2]/SuMdgA]]/SuMdgA2 | S1BC2S1 BC | | | |
| S2BC3S1 | [[(PI296341-FR/SuMdgA2)/SuMdgA2]/SuMdgA2]/SuMdgA2 | S2BC3 selfed | Fus2 screen | 48 | color |
| S3BC4 | [[[(PI296341-FR/SuMdgA2)/SuMdgA2]/SuMdgA2]/SuMdgA2]/SuMdgA2 | S2BC3S1 BC | | | color |
| S3BC4S1 | [[[(PI296341-FR/SuMdgA2)/SuMdgA2]/SuMdgA2]/SuMdgA2]/SuMdgA2 | S3BC4 selfed | Fus2 screen | | color |
| S3BC4S2 | [[[(PI296341-FR/SuMdgA2)/SuMdgA2]/SuMdgA2]/SuMdgA2]/SuMdgA2 | S3BC4S1 selfed | Fus2 screen | | |

Further Breeding Pedigrees

| LOT. | DESCRIPTION | PEDIGREE |
|---|---|---|
| 08FF5664-2 | F2 | L2/L1 |
| 08GH2279-5/2281-5 | F1 | L2/L1 |
| 09GH2968-12/2969-2 | BC1 | [(L2/L1)p12]L1 |
| 06GH1275-2 | S3 | L2 |
| 09GH2968-19/2969-3 | BC1 | [(L2/L1)p19]L1 |
| 09GH2968-81/2969-7 | BC1 | [(L2/L1)p81]/L1 |
| 09GH3214-2 | BC1S1 | [(L2/L1)p12]/L1 |
| 09GH3221-2 | BC1S1 | [(L2/L1)p19]/L1 |
| 06D7802-2 | F6 | L1 |
| 09GH3226-2 | BC1S1 | [(L2/L1)p81]/L1 |
| 10GH3562-6/3585 | S1BC2 | [[(L2/L1)p12]L1p6]/L1 |
| 10GH3578-1/3585 | S1BC2 | [[(L2/L1)p81]L1p1]/L1 |
| 12GH4827-1/4840 | S3BC4 | [[[[(L2/L1)p19]L1p9]/L1p14]/L1p1]/L1 |
| 11GH4359-9/4365 | S2BC3 | [[[(L2/L1)p81]L1p1]/L1p9]/L1 |
| 12D5606-1 | S3BC4S1 | [[[[(L2/L1)p19]L1p9]/L1p14]/L1p1]/L1p1 |
| 12D5606-2 | S3BC4S1 | [[[[(L2/L1)p19]L1p9]/L1p14]/L1p1]/L1p2 |
| 10GH3569-9/3585 | S1BC2 | [[(L2/L1)p19]L1p9]/L1 |
| 10GH3921-1 | S1BC2S1 | [[(L2/L1)p12]L1p6]/L1 |
| 10GH3937-1 | S1BC2S1 | [[(L2/L1)p19]L1p9]/L1 |
| 10GH3961-1 | S1BC2S1 | [[(L2/L1)p81]L1p1]/L1 |
| 11GH4347-4/4365 | S2BC3 | [[[(L2/L1)p12]L1p6]/L1p4]/L1 |
| 11GH4352-14/4365 | S2BC3 | [[[(L2/L1)p19]L1p9]/L1p14]/L1 |
| 11GH4352-4/4365 | S2BC3 | [[[(L2/L1)p19]L1p9]/L1p4]/L1 |
| 11GH4597-2 | S2BC3S1 | [[[(L2/L1)p12]L1p6]/L1p4]/L1 |
| 11GH4609-2 | S2BC3S1 | [[[(L2/L1)p19]L1p9]/L1p14]/L1 |
| 11GH4610-2 | S2BC3S1 | [[[(L2/L1)p19]L1p9]/L1p4]/L1 |
| 11GH4620-2 | S2BC3S1 | [[[(L2/L1)p81]L1p1]/L1p9]/L1 |
| 12D5601-2 | S3BC4S1 | [[[[(L2/L1)p12]L1p6]/L1p4]/L1p3]/L1p1 |
| 12D5611-1 | S3BC4S1 | [[[[(L2/L1)p19]L1p9]/L1p4]/L1p10]/L1p1 |
| 12D5611-2 | S3BC4S1 | [[[[(L2/L1)p19]L1p9]/L1p4]/L1p10]/L1p2 |
| 12D5635-2 | S3BC4S1 | [[[[(L2/L1)p81]L1p1]/L1p9]/L1p10]/L1p2 |
| 12D5638-1 | S3BC4S1 | [[[[(L2/L1)p81]L1p1]/L1p9]/L1p20]/L1p1 |
| 12GH4822-3/4840 | S3BC4 | [[[[(L2/L1)p12]L1p6]/L1p4]/L1p3]/L1 |
| 12GH4829-10/4840 | S3BC4 | [[[[(L2/L1)p19]L1p9]/L1p4]/L1p10]/L1 |
| 13GH5256-3 | S3BC4S2 | [[[[(L2/L1)p12]L1p6]/L1p4]/L1p3]/L1p1p3 |
| 13GH5256-4 | S3BC4S2 | [[[[(L2/L1)p12]L1p6]/L1p4]/L1p3]/L1p1p4 |
| 13GH5256-5 | S3BC4S2 | [[[[(L2/L1)p12]L1p6]/L1p4]/L1p3]/L1p1p5 |
| 13GH5256-6 | S3BC4S2 | [[[[(L2/L1)p12]L1p6]/L1p4]/L1p3]/L1p1p6 |
| 13GH5256-7 | S3BC4S2 | [[[[(L2/L1)p12]L1p6]/L1p4]/L1p3]/L1p1p7 |
| 13GH5256-8 | S3BC4S2 | [[[[(L2/L1)p12]L1p6]/L1p4]/L1p3]/L1p1p8 |
| 13GH5256-11 | S3BC4S2 | [[[[(L2/L1)p12]L1p6]/L1p4]/L1p3]/L1p1p11 |
| 13GH5257-2 | S3BC4S2 | [[[[(L2/L1)p19]L1p9]/L1p14]/L1p1]/L1p1p2 |
| 13GH5257-3 | S3BC4S2 | [[[[(L2/L1)p19]L1p9]/L1p14]/L1p1]/L1p1p3 |
| 13GH5257-4 | S3BC4S2 | [[[[(L2/L1)p19]L1p9]/L1p14]/L1p1]/L1p1p4 |
| 13GH5257-6 | S3BC4S2 | [[[[(L2/L1)p19]L1p9]/L1p14]/L1p1]/L1p1p6 |
| 13GH5257-7 | S3BC4S2 | [[[[(L2/L1)p19]L1p9]/L1p14]/L1p1]/L1p1p7 |
| 13GH5257-8 | S3BC4S2 | [[[[(L2/L1)p19]L1p9]/L1p14]/L1p1]/L1p1p8 |
| 13GH5258-4 | S3BC4S2 | [[[[(L2/L1)p19]L1p9]/L1p14]/L1p1]/L1p2p4 |
| 13GH5258-5 | S3BC4S2 | [[[[(L2/L1)p19]L1p9]/L1p14]/L1p1]/L1p2p5 |
| 13GH5258-6 | S3BC4S2 | [[[[(L2/L1)p19]L1p9]/L1p14]/L1p1]/L1p2p6 |
| 13GH5258-7 | S3BC4S2 | [[[[(L2/L1)p19]L1p9]/L1p14]/L1p1]/L1p2p7 |
| 13GH5258-9 | S3BC4S2 | [[[[(L2/L1)p19]L1p9]/L1p14]/L1p1]/L1p2p9 |
| 13GH5258-2 | S3BC4S2 | [[[[(L2/L1)p19]L1p9]/L1p14]/L1p1]/L1p2p2 |
| 13GH5258-3 | S3BC4S2 | [[[[(L2/L1)p19]L1p9]/L1p14]/L1p1]/L1p2p3 |
| 13GH5258-8 | S3BC4S2 | [[[[(L2/L1)p19]L1p9]/L1p14]/L1p1]/L1p2p8 |
| 13GH5259-2 | S3BC4S2 | [[[[(L2/L1)p19]L1p9]/L1p4]/L1p10]/L1p1p2 |
| 13GH5259-3 | S3BC4S2 | [[[[(L2/L1)p19]L1p9]/L1p4]/L1p10]/L1p1p3 |
| 13GH5259-4 | S3BC4S2 | [[[[(L2/L1)p19]L1p9]/L1p4]/L1p10]/L1p1p4 |
| 13GH5259-5 | S3BC4S2 | [[[[(L2/L1)p19]L1p9]/L1p4]/L1p10]/L1p1p5 |
| 13GH5259-6 | S3BC4S2 | [[[[(L2/L1)p19]L1p9]/L1p4]/L1p10]/L1p1p6 |
| 13GH5259-7 | S3BC4S2 | [[[[(L2/L1)p19]L1p9]/L1p4]/L1p10]/L1p1p7 |
| 13GH5259-8 | S3BC4S2 | [[[[(L2/L1)p19]L1p9]/L1p4]/L1p10]/L1p1p8 |
| 13GH5259-9 | S3BC4S2 | [[[[(L2/L1)p19]L1p9]/L1p4]/L1p10]/L1p1p9 |
| 13GH5260-1 | S3BC4S2 | [[[[(L2/L1)p19]L1p9]/L1p4]/L1p10]/L1p2p1 |
| 13GH5260-3 | S3BC4S2 | [[[[(L2/L1)p19]L1p9]/L1p4]/L1p10]/L1p2p2 |
| 13GH5260-4 | S3BC4S2 | [[[[(L2/L1)p19]L1p9]/L1p4]/L1p10]/L1p2p3 |
| 13GH5260-5 | S3BC4S2 | [[[[(L2/L1)p19]L1p9]/L1p4]/L1p10]/L1p2p4 |
| 13GH5260-6 | S3BC4S2 | [[[[(L2/L1)p19]L1p9]/L1p4]/L1p10]/L1p2p5 |
| 13GH5260-7 | S3BC4S2 | [[[[(L2/L1)p19]L1p9]/L1p4]/L1p10]/L1p2p6 |
| 13GH5260-8 | S3BC4S2 | [[[[(L2/L1)p19]L1p9]/L1p4]/L1p10]/L1p2p7 |
| 13GH5261-2 | S3BC4S2 | [[[[(L2/L1)p81]L1p1]/L1p9]/L1p10]/L1p2p2 |
| 13GH5261-3 | S3BC4S2 | [[[[(L2/L1)p81]L1p1]/L1p9]/L1p10]/L1p2p3 |
| 13GH5261-4 | S3BC4S2 | [[[[(L2/L1)p81]L1p1]/L1p9]/L1p10]/L1p2p4 |
| 13GH5261-5 | S3BC4S2 | [[[[(L2/L1)p81]L1p1]/L1p9]/L1p10]/L1p2p5 |
| 13GH5261-6 | S3BC4S2 | [[[[(L2/L1)p81]L1p1]/L1p9]/L1p10]/L1p2p6 |
| 13GH5261-7 | S3BC4S2 | [[[[(L2/L1)p81]L1p1]/L1p9]/L1p10]/L1p2p7 |
| 13GH5261-8 | S3BC4S2 | [[[[(L2/L1)p81]L1p1]/L1p9]/L1p10]/L1p2p8 |
| 13GH5262-1 | S3BC4S2 | [[[[(L2/L1)p81]L1p1]/L1p9]/L1p20]/L1p1p1 |
| 13GH5257-1 | S3BC4S2 | [[[[(L2/L1)p19]L1p9]/L1p14]/L1p1]/L1p1p1 |
| 13GH5258-1 | S3BC4S2 | [[[[(L2/L1)p19]L1p9]/L1p14]/L1p1]/L1p2p1 |
| 13GH5259-1 | S3BC4S2 | [[[[(L2/L1)p19]L1p9]/L1p4]/L1p10]/L1p1p1 |
| 13GH5261-1 | S3BC4S2 | [[[[(L2/L1)p81]L1p1]/L1p9]/L1p10]/L1p2p1 |
| 13GH5262-2 | S3BC4S2 | [[[[(L2/L1)p81]L1p1]/L1p9]/L1p20]/L1p1p2 |
| 12GH4836-10/4840 | S3BC4 | [[[[(L2/L1)p81]L1p1]/L1p9]/L1p10]/L1 |
| 12GH4836-20/4840 | S3BC4 | [[[[(L2/L1)p81]L1p1]/L1p9]/L1p20]/L1 |

NOTE:
A self at an earlier generation is not kept through the whole pedigree notation, but can be assumed e.g. there is a Self between each BC.
L2 is the PI used as a parent.
L1 is the recipient line used as a parent.

As a result, a “breeder level” Fon 2 resistant line with commercially acceptable fruit quality was created. To our knowledge this is a first. There are commercial pollinator types with Fon 2, but none are known to have commercial flesh quality or red fruit color.

### Example 2

### Screening Disease Resistance

Materials

1. Pathogen isolate/race: Fusarium oxysporum f sp. niveum
2. Isolate source:
Race 0 isolates include: Fon238-2
Race 1 isolates include: 03-15A, 811-B, F30
Race 2 isolates include: Cal.g.15(19), F137
   3. Long-term storage:
Suspension @ −80° C. in 15-20% glycerol. Desiccated culture on filter paper at 4° C.
   4. Susceptible controls:
Race 0: P8 (highly susceptible), Sugar Baby (highly susceptible)
Race 1: Include at least one highly susceptible and one intermediate susceptible.
Highly susceptible: P8, Sugar Baby
Intermediate susceptible: Charleston Gray, Charleston 76, Crimson Sweet
Race 2: Include at least one highly susceptible and one intermediate susceptible.
Highly susceptible: P8, Sugar Baby
Intermediate susceptible: Calhoun Gray, Dixie Lee, Charleston Gray, Charleston 76, Crimson Sweet. N.B. Calhoun Gray and Dixie Lee are also controls to make sure pathogen is race 2

### 5. Resistant Controls:

Race 0: Calhoun Gray, Charleston Gray, Charleston 76, Crimson Sweet, Dixie Lee, PI 296341-FR.
Race 1: Calhoun Gray, Dixie Lee, PI 296341-FR.
Race 2: PI 296341-FR.

### Method

### 1. Plant Culture:

Plant seeds in rows in shallow black flats filled with sand to allow greater root development; this growing method reduces transplant shock and mortality. Number of lines per flat: can grow 24 lines per flat if planting 12 seeds/line, 12 lines if planting 24 seeds/line and 7 lines if planting 48 seeds/line. A manageable size test is about 200 lines.

2. Inoculum Preparation:

Retrieve the targeted isolates from the suspension at −80° C. or the filter paper tubes in the refrigerator. Start culture on PDA with chlorophenicol (50 ppm) either by placing a small amount of frozen suspension or a piece of filter paper. Do not transfer cultures more than once; excessive transfers can lead to loss of pathogenicity.

Incubate cultures at ambient room temperature (20-24° C.) under 12-12 photoperiod for 5-10 days. Five days prior to the scheduled inoculation, start shake flask cultures of the various isolates in 500 ml of Fusarium liquid medium (see attached recipe from Esposito and Fletcher, 1961 in Appendix) and place on shaker 100 rpm room temperature until the day of inoculation. A few days later, start another set of shake flask cultures. The idea is to have inoculum of different ages, which seems to produce a more consistent result. Using a blender mix Fusarium liquid culture to make a thick slurry. Adjust spore count with a haemocytometer to 2-3×106 conidia/ml for race 1. For race 2, use a higher spore concentration (5×106 conidia/ml).

### 3. Inoculation Procedure:

Plants are ready for inoculation when cotyledons are fully expanded (about 9-14 days depending on the time of the year). Transplant shock damage is more severe when cotyledons are not fully expanded, so do not inoculate plants that are too young.

Pull the seedlings from each line and gently remove the sand by shaking and rinsing in a bucket of water. Pinch or cut roots off to approximately 1.5 cm (0.5 inch). Place the seedlings from each line together into a small plastic beaker containing approximately 30 ml of fresh inoculum and let sit for a minimum of five minutes. Do not reuse inoculum for inoculating another line. Collect used inoculum for autoclaving.

Transplant the inoculated seedlings into the pony pak insert (48 cells/flat) which have been filled with 1:1 sand: soil mix. Make one hole in each cell and place 2 seedlings/hole; 24 seedlings/line or plot #. Mist with water and provide shade by covering the test with Remay cloth (this fabric is normally used to protect plants from insect pests) to allow better recovery from transplant shock.

4. Conditions of Culture after Inoculation:

Flats are covered with Remay cloth for 48 hours. When watering is required carefully remove Remay cloth, water and then put back cloth in place. 3-5 days after inoculation count the number of seedlings/plot and record seedlings killed by transplant shock. This will ensure that dead plants counted after have been killed by the disease and not transplant shock.

Temperature in the greenhouse should be warm 26-29° C.

### Scoring

1. Performed at 14-18 days after inoculation
2. Description of symptoms scored:

Susceptible: cotyledons will begin to wilt and turn yellowish approximately 5 days after inoculation. Root system is usually dead and plant can be easily pulled from soil by a slight tug. Plants will often die out.

Resistant: plants that grow normally and remain lush green throughout the test. Root system continues to grow.

Intermediate: an intermediate rating can be given, especially when the test is not very severe and some plants are only slightly affected by the disease. A new root is sometimes generated from the base of a plant after transplanting while the primary root has died.

### 3. Ladder/Notation:

Plants are rated as resistant, intermediate or susceptible. Disease severity will vary between tests. On occasion, some resistant controls might show some stunting or mild symptoms.

### 4. Pictures of Symptoms:

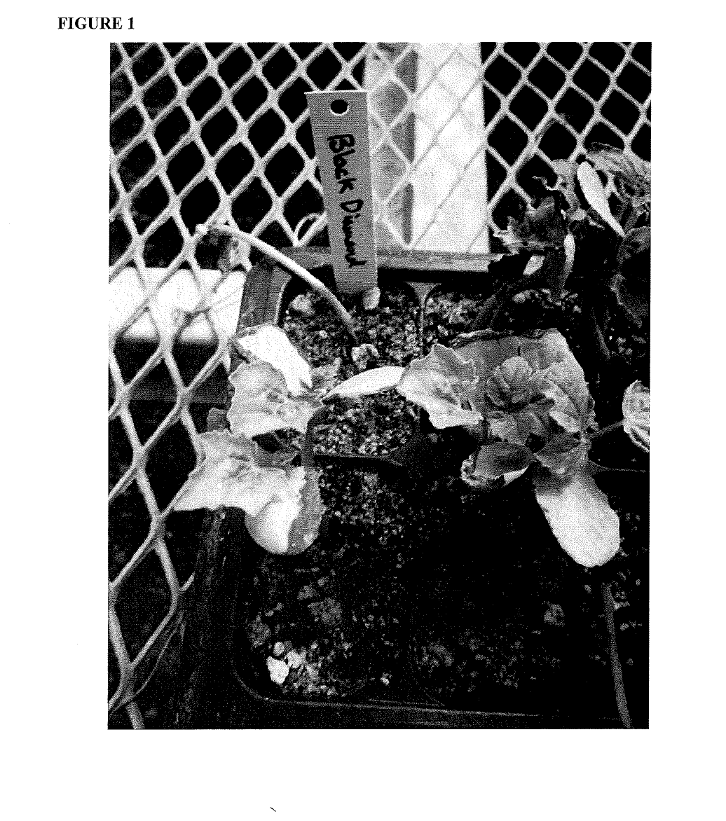

Example of early susceptible reaction with ‘Black Diamond’ is shown in FIG. 1. Example of resistant reaction from ‘Dixie Lee” to race 1 is shown in FIG. 2.

### APPENDIX

Recipe for Esposito and Fletcher (E/F) Fusarium liquid medium: Esposito R. and A. Fletcher. 1961. Arch. Biochem. Biophys. 93:369.

| | | |
|---|---|---|
| KH₂PO₄ | 1.5 | g |
| MgSO₄ | 0.25 | g |
| KNO₃ | 2.0 | g |
| Sucrose | 20.0 | g |
| FeCl₃ | 5.0 | mg |
| H₂O | 1000 | ml |

| | |
|---|---|
| pH | ~4.5 |

### Example 3

### Conferring Fon2 Resistance into Fon2-Susceptible Plants Via Non-Transgenic Methods

In one embodiment of the present invention, Fon2 resistance can be incorporated into a plant via non-transgenic (i.e., traditional) methods such as plant breeding. For example a cross can be made between a the Fon2 resistant plant of the present invention, and a second plant to produce a F1 plant. This F1 plant can then be subjected to multiple backcrossings to generate a near isogenic or isogenic line, wherein Fon2 resistance is integrated into the genetic background of the second plant.

### Deposit Information

A deposit of the watermelon seed of this invention is maintained by XXXXXXX. In addition, a sample of the Fon2 resistant watermelon seeds of this invention has been deposited with the National Collections of Industrial, Food and Marine Bacteria (NCIMB), 23 St Machar Drive, Aberdeen, Scotland, AB24 3RY, United Kingdom.

To satisfy the enablement requirements of 35 U.S.C. §112, and to certify that the deposit of the seeds of the present invention meets the criteria set forth in 37 C.F.R. §§1.801-1.809, Applicants hereby make the following statements regarding the deposited squash seed N9N030 (deposited as NCIMB Accession No. ______ on

1. During the pendency of this application, access to the invention will be afforded to the Commissioner upon request;

2. Upon granting of the patent the strain will be available to the public under conditions specified in 37 CFR 1.808;

3. The deposit will be maintained in a public repository for a period of 30 years or 5 years after the last request or for the enforceable life of the patent, whichever is longer;

4. The viability of the biological material at the time of deposit will be tested; and

5. The deposit will be replaced if it should ever become unavailable.

Access to this deposit will be available during the pendency of this application to persons determined by the Commissioner of Patents and Trademarks to be entitled thereto under 37 C.F.R. §1.14 and 35 U.S.C. §122. Upon allowance of any claims in this application, all restrictions on the availability to the public of the variety will be irrevocably removed by affording access to a deposit of at least 2,500 seeds of the same seed source with the NCIMB.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A watermelon plant resistance to F. oxysporum f. sp. niveum (Fon) race 2; wherein said watermelon plant has commercially acceptable fruit quality.

2. The watermelon plant of claim 1, wherein the watermelon plant is also resistant to F. oxysporum f. sp. niveum (Fon) race 0, Fon race 1 and/or Fon race 3.

3. The watermelon plant of claim 1, wherein the fruit flesh is red.

4. The watermelon plant of claim 1, wherein the fruit flesh is sweet with a Brix solid soluble content greater than 5.

5. The watermelon plant of claim 1, wherein the plant is a triploidy or tetraploidy plant.

6. (canceled)

7. The watermelon plant of claim 1, wherein the plant has an ultra-firm watermelon flesh phenotype.

8. A method of culturing plant tissue, plant part, plant organ, or cell culture comprising culturing at least part of the watermelon plant of claim 1 wherein said plant tissue, plant part, plant organ, or cell culture is cultured in conditions conducive to plant regeneration.

9-10. (canceled)

11. A method of producing watermelon plant comprising crossing the watermelon plant of claim 1 with another plant.

12-14. (canceled)

15. A method of breeding watermelon plants to produce altered pathogen tolerance and/or resistance while having commercially acceptable fruit quality comprising:
i) making a cross between the watermelon plant of claim 1 with a second watermelon plant to produce a F1 plant;
ii) backcrossing the F1 plant to the second plant; and
iii) repeating the backcrossing step one or more times to generate a near isogenic or isogenic line, wherein the near isogenic or isogenic line derived from the second plant with has conferred or enhanced Fon2 tolerance and/or resistance compared to that of the second plant prior to breeding, and has commercially acceptable fruit quality.

16. (canceled)

17. A method for producing a watermelon fruit comprising:
i) growing in a field a watermelon plant of claim 1;
ii) allowing said plant to set watermelon fruit; and
iii) harvesting said watermelon fruit.

18. (canceled)

19. A plant, a seed, a pollen, an ovule, a fruit, or a tissue culture of watermelon line, wherein the watermelon line is selected from the group consisting of:
(1) a watermelon line, wherein the representative seed of said line is having been deposited under NCIMB Accession No: ______; and
(2) a watermelon line, wherein the watermelon line has all the physiological and morphological characteristics of the watermelon line as described in (1).

20-24. (canceled)

25. A watermelon plant regenerated from the tissue culture of claim 19, wherein the regenerated plant has all the morphological and physiological characteristics of the watermelon plant of claim 19.

26. A method for producing a watermelon fruit, wherein the method comprises allowing pollination of a first watermelon plant and a second watermelon plant, wherein the first watermelon plant is the watermelon plant of claim 19.

27-28. (canceled)

29. A method for producing seeds of a watermelon plant, wherein the method comprises the steps of: a) growing in a field the watermelon plant according to claim 19; b) conducting pollination of said plant with the same or a different plant; and c) harvesting seed of said plant.

30. A method for producing a hybrid watermelon variety, wherein the method comprises the steps of: a) planting in a field a first and a second watermelon plant, wherein said first watermelon plant is the male parent, wherein said second watermelon plant is the female parent, and wherein said first or said second watermelon plant is the watermelon plant according to claim 19; b) conducting pollination between said first and second watermelon plants; and c) harvesting seed from said female parent, wherein said seed is seed of a hybrid watermelon variety.

31. The method of claim 30 wherein step (c) comprises identifying plants resistant to F. oxysporum f. sp. niveum (Fon) race 2.

32. The method of claim 30 wherein step (c) comprises identifying plants having commercially acceptable fruit quality.

33-35. (canceled)

36. A method of introducing one or more desired traits into the watermelon plant of claim 19 wherein the method comprises:
a) crossing the watermelon plant of claim 19 with plants of another watermelon line that comprise one or more desired traits to produce progeny plants, b) selecting progeny plants that have the one or more desired traits to produce selected progeny plants;
c) crossing the selected progeny plants with the watermelon plant of claim 19 to produce backcross progeny plants;
d) selecting for backcross progeny plants that have the one or more desired traits and physiological and morphological characteristics of the watermelon plant of claim 19 to produce selected backcross progeny plants; and
e) repeating steps (c) and (d) one or more times in succession to produce selected second or higher backcross progeny plants that comprise the desired one or more trait and the physiological and morphological characteristics of the watermelon plant of claim 19.

37. A watermelon plant produced by the method of claim 36, wherein the plant has the one or more desired traits and all of the physiological and morphological characteristics of the watermelon plant of claim 19.

38. The method of claim 29, further comprising growing the resultant seeds to produce one or more watermelon plant progeny.

39. A watermelon plant progeny produced by the method of claim 38, wherein the progeny plants are resistance to F. oxysporum f. sp. niveum (Fon) race 2, and having commercially acceptable fruit quality.

40. (canceled)
